(19) United States
(12) Patent Application Publication
(10) Pub. No.: US 2023/0416265 A1
(43) Pub. Date: Dec. 28, 2023

(54) COMPOUNDS FOR THE DETECTION OF HEME OXYGENASE 1 (HO-1), AND METHODS AND USES INVOLVING THE SAME

(71) Applicant: IMPERIAL COLLEGE INNOVATIONS LIMITED, London (GB)

(72) Inventors: Joseph James BOYLE, London (GB); Nicholas J. LONG, London (GB); Edward R.H. WALTER, London (GB); Ying GE, London (GB); Justin C. MASON, London (GB)

(21) Appl. No.: 18/252,597

(22) PCT Filed: Nov. 12, 2021
(86) PCT No.: PCT/GB2021/052929

§ 371 Date: May 11, 2023

(30) Foreign Application Priority Data
Nov. 12, 2020 (GB) 2017871.1

(51) Int. Cl. C07D 487/22; A61K 49/00

(52) U.S. Cl. CPC C07D 487/22; A61K 49/0034; A61K 49/0036

(57) ABSTRACT

tion of heme oxygenase 1 (HO-1), in particular porphyrin, chlorin, bacteriochlorin or isobacteriochlorin compounds of formula I as defined herein having a tetrapyrrole or reduced tetrapyrrole backbone and a fluorophore. Such compounds can be used in the detection of HO-1 in vivo, ex vivo and in vitro, and can also be used in methods of diagnosis and as research reagents.

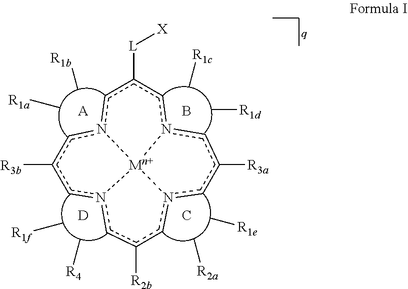

### FIELD OF THE INVENTION

The present invention relates to compounds for the detection of heme oxygenase 1 (HO-1), in particular porphyrin, chlorin, bacteriochlorin or isobacteriochlorin compounds having a tetrapyrrole or reduced tetrapyrrole backbone and a fluorophore. Such compounds can be used in the detection of HO-1 in vivo, ex vivo and in vitro, and can also be used in methods of diagnosis and as research reagents.

### BACKGROUND

Heme oxygenase (HO) is an important homeostatic microsomal enzyme in vascular biology and cell signalling. The primary role of HO is to prevent the accumulation of cytotoxic ‘free’ heme (Fe-PPIX), which has the potential to act as a Fenton catalyst in vivo, leading to the generation of reactive oxygen species.

Heme catabolism by HO is a three-step reaction that requires molecular oxygen and NADPH cytochrome p450 reductase. During the process, the porphyrin ring is regio-selectively de-cyclised at the 5 position (also known as the α-carbon atom) to form α-biliverdin, with the loss of carbon monoxide (CO) and ferrous iron (Fe²⁺). This is shown in Scheme 1. Biliverdin is further converted to bilirubin by biliverdin reductase in the cytosol.

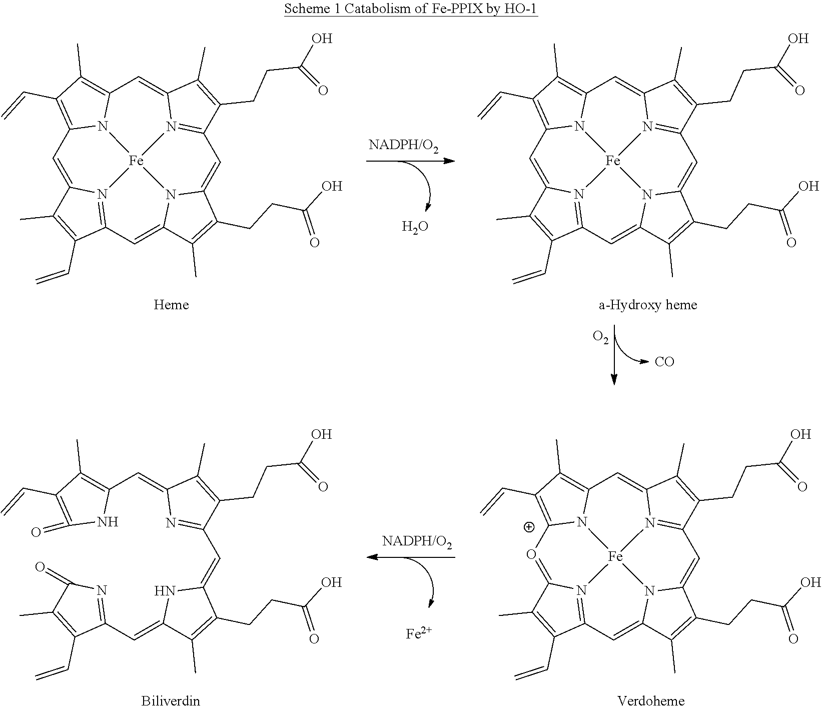

Each of the by-products of heme degradation is known to be cytoprotective. For example, Fe²⁺ is chelated by ferritin, where it is safely stored out of Fenton activity and a number of studies have demonstrated that CO is an essential intracellular signalling molecule, activating the KATP ion channel and suppresses endothelial cell apoptosis. Finally, bilirubin has also been proven to display antioxidant properties. Individuals with bilirubin concentrations above basal levels, in genetic disorders such as Gilbert syndrome, are significantly less likely to develop cardiovascular disease over their lifetime.

There are two main isoforms of HO, namely heme oxygenase-1 (HO-1) and heme oxygenase-2 (HO-2). Both isoforms of HO catalyse such heme degradation. HO-1 is the inducible isoform of the enzyme that degrades heme. The other isoenzyme, HO-2, is found at low levels and is not heme-inducible. Thus, HO-1 is the only isoform that is induced by cellular stress stimuli in the body, for example, after intraplaque haemorrhage (IPH), and has a defensive role in a number of diseases including atherosclerosis and cancer. For this reason, HO-1 is a critical enzyme for vascular health and disease and a marker of hemorrhage in ruptured coronary plaques and hemorrhagic stroke.

Although HO-1 activity can be quantified, all existing assays of HO-1 activity are destructive, labour-intensive, semi-quantitative, unreliable, low throughput and incompatible with real-time measurements on live cells. Moreover, these assays almost always detect protein levels rather than activity of HO-1 and do not detect HO-1 on a single-cell basis. The typical assay currently in use in the art is decades old and involves cell lysis in a complex mix of unstable enzymatic cofactors. After the reaction, the mix is extracted in chloroform and measured spectrophotometrically. This process is described in: Tenhunen R, Marver H S, Schmid R. “The enzymatic conversion of hemoglobin to bilirubin”, Trans Assoc Am Physicians, 1969;82:363-71; and Pimstone N R, Tenhunen R, Seitz P T, Marver H S, Schmid R. “The enzymatic degradation of hemoglobin to bile pigments by macrophages”, J Exp Med 1971 Jun. 1,133(6):1264-81. Kinetics and localisation for HO-1 are particularly difficult, with in vivo dynamic application being impracticable. These problems impede the elucidation of the role of HO-1 in pathophysiology and in pharmacology, and clinical translation, particularly in vivo.

Thus, there remains a need for a reliable HO-1 detection method for live cells or tissues. The development of a chemical probe for detecting HO-1 would also be of particular use in the detection of, for instance, IPH and associated diseases, importantly, before the on-set of more severe associated conditions.

### SUMMARY OF THE INVENTION

It has surprisingly been found that through the modification of a porphyrin, chlorin, bacteriochlorin or isobacteriochlorin with a fluorophore at the 5-position, the concentration and/or location of HO-1 may be observed, as HO-1 acts on such molecules, cleaving the fluorophore from the tetrapyrrole or reduced tetrapyrrole backbone. This allows for the monitoring of conditions characterised by HO-1 over-expression, such as intraplaque haemorrhage, acute coronary syndrome and stroke caused by intraplaque haemorrhage and atherosclerosis.

This effect is particularly beneficial when the tetrapyrrole or reduced tetrapyrrole backbone of the porphyrin, chlorin, bacteriochlorin or isobacteriochlorin and the fluorophore represent a Fluorescence Resonance Energy Transfer (FRET) pair, as fluorescence from the fluorophore is modulated (i.e. increased or decreased) when HO-1 acts on the compound. This confirms that HO-1 is present at the location of fluorescence modulation, allows for more sensitive detection of HO-1, and may allow for determination of the concentration of HO-1.

The compounds of the present invention are extremely versatile and may be used in in vivo, ex vivo and in vitro diagnoses of a disease. In addition, they may be used as research reagents, for example to determine the activity and/or presence of HO-1.

Accordingly, in a first aspect, the present invention provides a compound represented by Formula I or a pharmaceutically acceptable salt thereof:

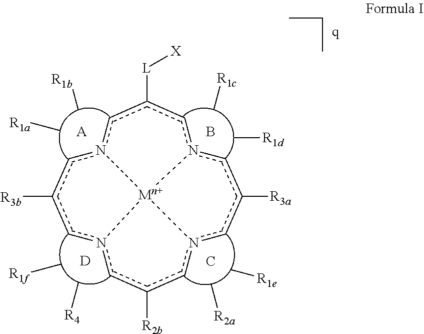

wherein the compound is selected from porphyrins, chlorins, bacteriochlorins or isobacteriochlorins, and wherein:
[symbol] represents a double or single bond;
[symbol] represents a dative or covalent bond between nitrogen and Mⁿ⁺,
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₑ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH2, —OH, —SH, —NH₂, —COORS, —CSOR₅, —COSR₅, —CSSR₅, —CON(R₅)₂, —CONHR₅, —OR ₅, —SRS, —N(R₅)₂, —NHR₅, —CH(OR₅)₂, —CH(OR₅)(SR₅), —CH(SR₅)₂,

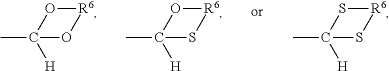

wherein R₅ is monovalent C₁ to C₅-hydrocarbyl or X2, and R₆ is C₁ to C₅alkylene; or R₂ₐ is taken together with R₁ₑ to form a cyclocarbyl;
R₁ₐ and R_{3b} are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR₇), —CH(SR₇)₂,

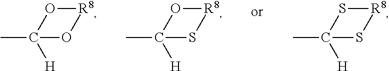

wherein R₇ is monovalent C₁ to C₅-hydrocarbyl or X3, and R₈ is C₁ to C₅-alkylene,
L is a linker;
X, X2 and X3 are fluorophores, which may be the same or different;
Mⁿ⁺ is a cation having a positive charge of integer n, wherein n is from 1 to 3;
q is the overall charge of the compound; and
A, B, C and D each independently represent a five-membered nitrogen-containing heterocycle selected from pyrrole, pyrroline or pyrrolidine rings.

In embodiments, the fluorophore X and the tetrapyrrole or reduced tetrapyrrole backbone of the porphyrin, chlorin, bacteriochlorin or isobacteriochlorin represent a FRET pair.

In embodiments, fluorescence from X is quenched in the compound of Formula I following excitation of X. In alternative embodiments, fluorescence from X is observed in the compound of Formula I following excitation of the tetrapyrrole or reduced tetrapyrrole backbone of the porphyrin, chlorin, bacteriochlorin or isobacteriochlorin. These effects may result from FRET pairing of the tetrapyrrole or reduced tetrapyrrole backbone and fluorophore X.

In another aspect, there is provided a compound according to the present invention for use in a method of diagnosis, optionally of a disease characterised by heme oxygenase-1 (HO-1) over-expression.

In a further aspect, there is provided a compound according to the present invention for use in a method of diagnosis in vivo of intraplaque haemorrhage, acute coronary syndrome and/or stroke caused by intraplaque haemorrhage and/or atherosclerosis.

In an aspect there is also provided a compound according to the present invention for use in a method of treatment of acute coronary syndrome and/or stroke caused by intraplaque haemorrhage and/or atherosclerosis.

In an additional aspect, there is provided a method for in vitro and/or ex vivo diagnosis of intraplaque haemorrhage, acute coronary syndrome and/or stroke caused by intraplaque haemorrhage and/or atherosclerosis using the compound according to the present invention.

In a further aspect, there is provided a method of imaging heme oxygenase 1 and/or intraplaque haemorrhage using a compound according to the present invention.

In an aspect, there is also provided use of a compound according to the present invention in the in vitro and/or ex vivo diagnosis of intraplaque haemorrhage, acute coronary syndrome and/or stroke caused by intraplaque haemorrhage and/or atherosclerosis.

In an aspect, there is further provided use of a compound according to the present invention as a contrast agent for imaging heme oxygenase 1 and/or intraplaque haemorrhage.

In an aspect, there is also provided use of a compound according to the present invention as a research reagent, preferably for the detection of heme oxygenase 1.

In an additional aspect, there is provided a method of preparing a compound according to the present invention, comprising reacting a compound of Formula Vla to Vlc or a pharmaceutically acceptable salt thereof:

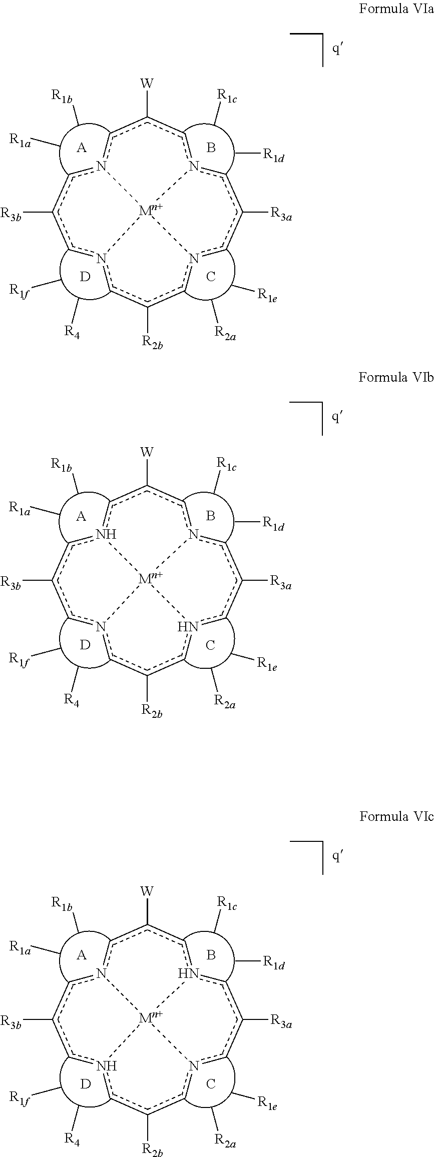

wherein the compound is a porphyrin, chlorin, bacteriochlorin or isobacteriochlorin, and wherein:
[symbol] represents a double or single bond;
[symbol] represents a dative or covalent bond between nitrogen and Mⁿ⁺,
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COOR₅, —CSOR₅, —COSR ₅, —CSSR₅, —CON(R₅)₂, —CONHR₅, —OR₅, —SRS, —N(R₅)₂, —NHR₅, —CH(OR₅)₂, —CH(OR₅)(SR₅), —CH(SR₅)₂,

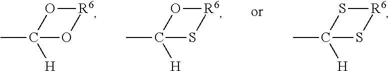

wherein R₅ is monovalent C₁ to C₅-hydrocarbyl or X2, and R₆ is C₁ to C₅ alkylene; or R₂ₐ is taken together with R₁ₑ to form a cyclocarbyl;
R₃ₐ and R_{3b} are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR₇), —CH(SR₇)₂,

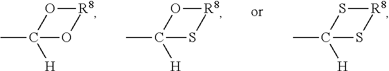

wherein R₇ is monovalent C₁ to C₅-hydrocarbyl or X3, and R₈ is C₁ to C₅-alkylene,
L is a linker;
X2 and X3 are fluorophores which may be the same or different;
Mⁿ⁺ is a cation having a positive charge of integer n, wherein n is from 1 to 3;
q′ is the overall charge of the compound;
A, B, C and D each independently represent a five-membered nitrogen-containing heterocycle selected from pyrrole, pyrroline or pyrrolidine rings; and
W comprises an azide or an alkyne functional group,
   with a compound having the formula [Z-X]^{q″}, wherein X is a fluorophore which may be the same as or different from X2 and X3, q″ is the overall charge of the compound, and Z comprises an azide or an alkyne functional group, wherein one of W and Z comprises an azide functional group and the other comprises an alkyne functional group.

### BRIEF DESCRIPTION OF THE DRAWINGS

The present inventions will now be described by way of example and with reference to the accompanying Figures in which:

FIG. 1 illustrates the absorbance spectra of 1, 1 h, 1g and 1f in PBS buffer at pH 7.4;

FIG. 2 illustrates the absorbance spectra of C3, C3g and 1f in PBS buffer at pH 7.4;

FIG. 3 illustrates the absorbance and emission spectra of 1f and the absorbance of 1e in PBS buffer at pH 7.4;

FIG. 4 illustrates the emission spectra of 1g, 1 h and 1 in PBS at pH 7.4;

FIG. 5 illustrates the emission spectra of 1g, 1 h and 1 in chloroform;

FIG. 6 illustrates the emission spectra of C3 and C3g in PBS at pH 7.4;

FIG. 7 illustrates the emission spectra of C3 and C3g in chloroform;

FIG. 8 illustrates the emission spectrum of 1f compared to 1 in PBS at pH 7.4.

FIG. 9 illustrates the quenching of fluorescence of 1i upon the addition of HO-1 in E coli lysates.

FIG. 10 illustrates the fluorescence of 1 in the presence of HO-1 under UV excitation with (top) and without (bottom) the addition of NADPH;

FIG. 11 shows the change in coumarin emission intensity following incubation of 1 and of C3 with HO-1 in E. coli lysates with and without NADPH, following acidification and extraction into chloroform. Photophysical measurements were recorded in PBS at pH 7.4;

FIG. 12 shows the average change in fluorescence emission intensity following incubation of 1 and of C3 with HO-1 in E. coli lysates with and without NADPH as a bar chart. Error bars are calculated as standard deviations of the repeats;

FIG. 13 illustrates the metabolism of 1 followed by coumarin 1i emission intensity;

FIG. 14 illustrates the absorbance spectra of 1 (A) and C3 (B) in E coli lysates overexpressing HO-1 with and without incubation with NADPH and biliverdin as a reference;

FIG. 15 shows the LCMS trace of 1i superimposed over LCMS traces of 1 (A) and C3 (B) at 320 nm in E. coli lysates in the presence of HO-1, both with and without the addition of NADPH;

FIG. 16 shows the MALDI-MS spectrum of 1 in the presence of HO-1 and NADPH;

FIG. 17 shows the MALDI-MS spectrum of C3 in the presence of HO-1 and NADPH;

FIG. 18 illustrates the toxicity of 1, 1i, heme, iron and biliverdin at a range of concentrations in RAW cells; and

FIG. 19 illustrates the fluorescence activity in arbitrary fluorescent units (AFU) of compounds 1 and C3 in cultured human blood-derived inflammatory cells.

### DETAILED DESCRIPTION OF THE INVENTION

For the purposes of the present invention, the following terms as used herein shall, unless otherwise indicated, be understood to have the following meanings. Other terms that are not as defined below are to be understood as their normal meaning in the art.

Porphyrins, chlorins, bacteriochlorins and isobacteriochlorins comprise a tetrapyrrole or reduced tetrapyrrole ring system, in which four subunits selected from pyrrole, pyrroline or pyrrolidine rings are interconnected at the C2 and C5 position (also known as the alpha-position) via methine bridges (═CH—) to form a ring.

The 1 to 24 numbering scheme for the carbon and nitrogen atoms in the tetrapyrrole or reduced tetrapyrrole ring system in porphyrins and reduced porphyrins is used throughout this application, unless stated otherwise. This accepted numbering scheme is described in “IUPAC, Compendium of Chemical Terminology”, 2nd ed. (the “Gold Book”), compiled by McNaught, A. D. and Wilkinson, A., 1997 Blackwell Scientific Publications, Oxford (Online version (2019-present) created by Chalk, S. J., ISBN 0-9678550-9-8. https://doLorq/10.1351/goldbook), and Moss, G. P., “Nomenclature of tetrapyrroles, Recommendations 1986 IUPAC-IUB Joint Commission on Biochemical Nomenclature (JCBN)”, Eur J Biochem., 1988, 178(2), 277-328. In this numbering scheme, porphyrins are numbered as follows:

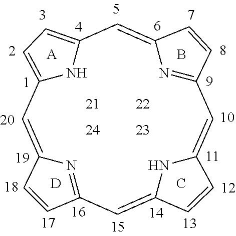

This numbering system can also be applied to reduced porphyrins. In this numbering system, the 2, 3, 7, 8, 12, 13, 17 and 18 positions can be referred to generally as ‘beta-positions’ (i.e. of the pyrrole rings) or ‘non-fused carbon atoms’. Similarly, positions at 1, 4, 6, 9, 11, 14, 16 and 19 can be referred to generally as ‘alpha-positions’ or ‘fused carbon atoms’, while those at 5, 10, 15 and 20 can be referred to generically as ‘meso-positions’. In the alternative Fischer numbering system, the 5 position is referred to as the α-position.

For nomenclature purposes, the saturated nitrogen atoms are generally at positions 21 and 23. However, the tetrapyrrole or reduced tetrapyrrole ring system is tautomeric with respect to the location of these two hydrogen atoms and thus they may be associated with any two opposing nitrogen atoms, for example, the saturated nitrogen atoms may be at positions 22 and 24. Herein, disclosure of one tautomer is considered to disclose all tautomers of that structure which result from the movement of the position of these saturated nitrogen atoms, unless explicitly stated otherwise. Typically, tautomers can readily interconvert, commonly through relocation of a proton or a very small number of double and single bonds.

The structures herein are drawn as a single resonance form. However, conjugated structures may exist in alternative resonance forms.

As described in more detail herein, the tetrapyrrole or reduced tetrapyrrole backbone may include substituents. Substituents may be at the 2, 3, 7, 8, 12, 13, 17 and/or 18 positions and/or the 5, 10, 15 and/or 20 positions. When the compound is a porphyrin and the tetrapyrrole backbone is substituted (i.e. at least one of positions 2, 3, 7, 8, 12, 13, 17, 18, 10, 15 and 20 is bound to a moiety other than H) and the substitution of rings A to D is not identical two tautomers of the structure will be present. When the compound is a chlorin, bacteriochlorin or isobacteriochlorin and the reduced tetrapyrrole backbone is substituted (i.e. at least one of positions 2, 3, 7, 8, 12, 13, 17, 18, 5, 10, 15 and 20 is bound to a moiety other than H) and the substitution of rings A to D is not identical, multiple isomers of the compound may be present. Each of these isomers may in turn have a tautomer. These structures will be discussed in further detail below. The isomers shown are drawn on the assumption that each of A to D is not identical. However, if some of rings A to D are identical, the overall number of possible isomers discussed below will be reduced.

Upon coordination of porphyrins to a cation, the two hydrogen atoms bound to the saturated nitrogen atoms (positions 21 and 23 in the description above) are deprotonated, resulting in the tetrapyrrole or reduced tetrapyrrole backbone having a formal -2 charge. These negatively charged nitrogen atoms form a covalent bond with the cation. Meanwhile the other two nitrogen atoms (positions 22 and 24 above) form a dative bond with the cation through donation of their lone pair. This is also true of chlorins, bacteriochlorins or isobacteriochlorins, which are discussed in further detail below.

‘Porphyrins’ as used herein refers to a group of heterocyclic macrocycle organic compounds, comprising a cyclic tetrapyrrole backbone (i.e. the backbone comprises four modified pyrrole subunits). This structure is conjugated, comprising an 18π-electron aromatic pathway.

Porphyrins are tautomeric with respect to the location of the two hydrogen atoms not involved in this conjugated system. The two tautomers of unsubstituted porphyrins (which is commonly called porphyrin) are as shown in Table 1.

TABLE 1

| Tautomer 1 | Tautomer 2 |
|---|---|
| 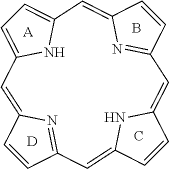 | 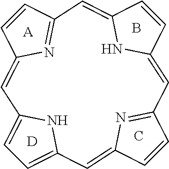 |

Although these tautomers are in fact identical molecules in this example (as a result of symmetry in the structure), as will be appreciated, this may not be true when rings A to D are non-identical (e.g. if the molecule is substituted non-symmetrically about the tetrapyrrole backbone).

‘Chlorins’ as used herein refer to reduced porphyrins (dihydroporphyrins) in which saturated carbon atoms are located at the non-fused carbon atoms of one of the pyrrole rings.

Theoretically, chlorins may comprise a reduced tetrapyrrole backbone comprising three pyrrole subunits and a pyrroline subunit, depending on which ring is reduced. These structures contain an 18π-electron aromatic pathway. Preferably the aromatic structure is retained, therefore the reduced tetrapyrrole backbone preferably comprises three pyrrole subunits and a pyrroline subunit, maintaining the 18π-electron aromatic pathway.

The two possible tautomers of unsubstituted chlorins (commonly called chlorin) which retain the aromatic pathway are shown in Table 2.

TABLE 2

| Tautomer 1 | Tautomer 2 |
|---|---|
| 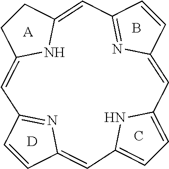 | 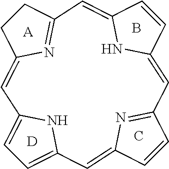 |

In both of these structures, ring A has been reduced, however the conjugation in the rest of the backbone differs. Both tautomers include an 18π-electron aromatic pathway. However, the 18π-electron aromatic pathway of tautomer 1 incorporates the nitrogen lone-pair of pyrroline A which becomes delocalised within the aromatic system, thus reducing its aromatic character compared to tautomer 2. Therefore, tautomer 2 is preferred. In order to maintain this 18π-electron aromatic pathway, only pyrroles having an unsaturated nitrogen atom (e.g. positions 22 and 24 in the above numbered representation) can be reduced, meaning that tautomers which would result from the reduction of pyrroles having saturated nitrogen atoms (e.g. positions 21 and 23 above) are less preferred.

Chlorins may comprise multiple isomers if rings A to D are non-identical (e.g. if the molecule is substituted non-symmetrically about the reduced tetrapyrrole backbone) depending on which pyrrole ring is reduced. Thus, chlorins may include the structures in Table 3. Note, only those structures maintaining the 18π-electron aromatic pathway have been included.

TABLE 3

| Ring this is reduced | |
|---|---|
| A | 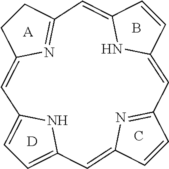 |
| B | 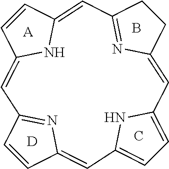 |
| C | 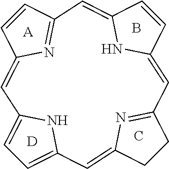 |
| D | 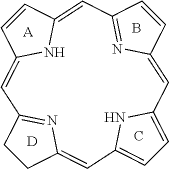 |

These structures are based on the assumption that none of rings A to D are identical. However, depending on the substitution pattern on rings A to D, some of these structures may be identical.

‘Bacteriochlorins” as used herein refer to reduced porphyrins (tetrahydroporphyrins) in which saturated carbon atoms are located at the non-fused carbon atoms of two diagonally opposite pyrrole rings. Thus, the reduced tetrapyrrole backbone maintains the 18π-electron aromatic pathway.

Bacteriochlorins comprise a reduced tetrapyrrole backbone comprising two pyrrole subunits and two pyrroline subunits.

Due to the requirement that non-fused carbon atoms of diagonally opposite rings are reduced, only pyrroles having an unsaturated nitrogen atom (e.g. positions 22 and 24 in the above representation) can be reduced as these both include double bonds between the non-fused carbon atoms and are diagonally opposite one another. This is not true of pyrroles having saturated nitrogen atoms (e.g. positions 21 and 23 above). Thus, tautomers cannot be formed and are not included in further discussion.

Bacteriochlorins may comprise multiple isomers if rings A to D are not identical (e.g. if the molecule is substituted non-symmetrically about the reduced tetrapyrrole backbone) depending on which pair of pyrrole rings are reduced. Thus, the possible structures of unsubstituted bacteriochlorins (commonly called bacteriochlorin) are shown in Table 4.

TABLE 4

| Rings that are reduced | |
|---|---|
| A and C | 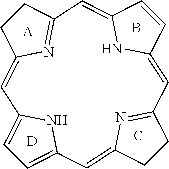 |
| B and D | 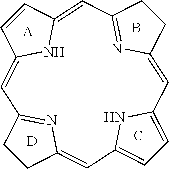 |

These structures are based on the assumption that none of rings A to D are identical.

However, depending on the substitution pattern on rings A to D, some of these structures may be identical.

‘Isobacteriochlorins’ as used herein refer to reduced porphyrins (tetrahydroporphyrins) in which saturated carbon atoms are located at the non-fused carbon atoms of two adjacent pyrrole rings. Thus, the reduced tetrapyrrole backbone comprises a 16π-electron conjugated system and are not aromatic.

lsobacteriochlorins comprise a reduced tetrapyrrole backbone comprising two pyrrole subunits and one pyrroline subunit and one pyrrolidine subunit.

lsobacteriochlorins may comprise multiple isomers if rings A to D are not identical (e.g. if the molecule is substituted non-symmetrically about the reduced tetrapyrrole backbone) depending on which pair of pyrrole rings are reduced. Each of these structures may exist as tautomers. Thus, unsubstituted isobacteriochlorins (commonly called isobacteriochlorin) may include the structures in Table 5.

TABLE 5

| Rings that | | |
|---|---|---|
| are reduced | Tautomer 1 | Tautomer 2 |
| A and B | 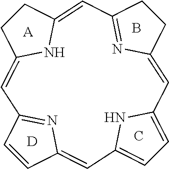 | 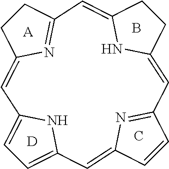 |
| B and C | 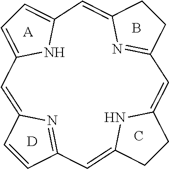 | 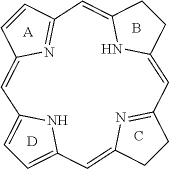 |
| C and D | 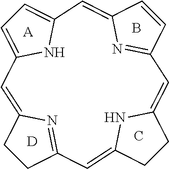 | 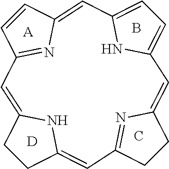 |
| D and A | 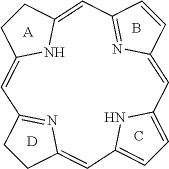 | 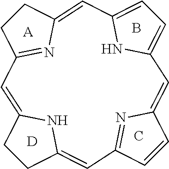 |

These structures are based on the assumption that none of rings A to D are identical. However, depending on the substitution pattern on rings A to D, some of these structures may be identical.

The term ‘hydrocarbyl’ as used herein refers to a monovalent or divalent group comprising a major portion of hydrogen and carbon atoms, preferably consisting exclusively of hydrogen and carbon atoms, which group may be aromatic or aliphatic, preferably aliphatic, saturated or unsaturated, preferably saturated, branched or unbranched, and containing from 1 to 20 atoms (C1 to C20), preferably 1 to 12 atoms (C1 to C12), more preferably 1 to 6 atoms (C1 to C6). In preferred embodiments, the hydrocarbyl group is an aliphatic group containing from 1 to 20 carbon atoms.

The hydrocarbyl group may be entirely aliphatic or a combination of aliphatic and aromatic portions. Examples of hydrocarbyl groups therefore include acyclic groups, as well as groups that combine one or more acyclic portions and one or more cyclic portions, which may be selected from cyclic alkyl and aryl groups.

Examples of aliphatic hydrocarbyl groups include acyclic groups, non-aromatic cyclic groups and groups comprising both an acyclic portion and a non-aromatic cyclic portion. These include alkyl, alkylene, alkenyl, alkynyl and carbocyclyl (e.g. cycloalkyl or cycloalkenyl) groups. Examples of aromatic hydrocarbyl groups include aryl and heteroaryl groups.

Additionally or alternatively, one or more of the carbon atoms, and any substituents attached thereto, of the hydrocarbyl group may be replaced with an oxygen atom (—O—), a nitrogen atom (—NR—), wherein R is H or an alkyl group, or a sulphur atom (—S—), preferably an oxygen atom, provided that the oxygen, nitrogen or sulphur atom is not bonded to another heteroatom. In such an embodiment, the hydrocarbyl group may comprise an ether, an amine, or a thioether. In embodiments, up to 20% of the chain atoms are heteroatoms, preferably up to 10%. In other embodiments, there are between 1 and 3 heteroatoms present in the chain, preferably 1 or 2, most preferably 1.

The hydrocarbyl group may be substituted by one or more groups, for example, alkyl groups comprising one or more heteroatoms, aryl or heteroaryl groups or combinations thereof. The hydrocarbyl group may be substituted by one or more groups that are preferably selected from hydroxyl (—OH) groups, carboxylic acid (—COOH) groups and C1 to C5 esters (—COO(C1 to C5 alkyl)), more preferably selected from carboxylic acid (—COOH) groups and C1 to C5 esters (—COO(C1 to C5 alkyl)), and most preferably carboxylic acid (—COOH) groups.

The term ‘cyclocarbyl’ is used herein to refer to a cyclic hydrocarbyl, wherein the hydrocarbyl is as defined above. The cyclocarbyl may comprise one or more double bonds. The ring may or may not be aromatic. The heterocycle may contain from 3 to 10 carbon atoms and may optionally have alkyl groups attached thereto. Examples of heterocyclic groups include groups containing from 3 to 8 carbon atoms, e.g. from 4 to 6 carbon atoms.

The term ‘heterocycle’ as used herein refers to a cyclocarbyl comprising one or more heteroatoms, preferably selected from nitrogen, oxygen and sulphur. The heterocycle may comprise one or more double bonds. The ring may or may not be aromatic. The heterocycle may contain from 3 to 10 carbon atoms and may optionally have alkyl groups attached thereto. Examples of heterocyclic groups include groups containing from 3 to 8 carbon atoms, e.g. from 4 to 6 carbon atoms. Particular examples include heterocyclic groups containing 3, 4, 5 or 6 ring carbon atoms. Heterocyclic groups comprising nitrogen include pyrrole, pyrroline or pyrrolidine rings.

The term ‘alkyl’ as used herein refers to a monovalent straight- or branched-chain hydrocarbyl group consisting exclusively of hydrogen and carbon atoms containing from 1 to 20 carbon atoms. Examples of alkyl groups include alkyl groups containing from 1 to 20 carbon atoms (C1 to C20), preferably 1 to 12 atoms (C1 to C12), more preferably 1 to 6 atoms (C1 to C6). Particular examples include alkyl groups containing 1, 2, 3, 4, 6, 8, 10, 12 or 14 carbon atoms. Unless specifically indicated otherwise, the term ‘alkyl’ does not include optional substituents.

The term ‘alkylene’ as used herein refers to a divalent straight- or branched-chain hydrocarbyl group consisting exclusively of hydrogen and carbon atoms and containing from 1 to 20 carbon atoms. Examples of alkyl groups include alkyl groups containing from 1 to 20 carbon atoms (C1 to C20), preferably 1 to 12 atoms (C1 to C12), more preferably 1 to 6 atoms (C1 to C6). Particular examples include alkylene groups that contain 1, 2, 3, 4, 5 or 6 carbon atoms. Unless specifically indicated otherwise, the term ‘alkylene’ does not include optional substituents.

The term ‘cycloalkyl’ as used herein refers to a monovalent saturated aliphatic hydrocarbyl group containing from 3 to 20 carbon atoms and containing at least one ring, wherein said ring has at least 3 ring carbon atoms. The cycloalkyl groups mentioned herein may optionally have alkyl groups attached thereto. Examples of cycloalkyl groups include cycloalkyl groups containing from 3 to 16 carbon atoms, e.g. from 3 to 10 carbon atoms. Particular examples include cycloalkyl groups containing 3, 4, 5 or 6 ring carbon atoms. Examples of cycloalkyl groups include groups that are monocyclic, polycyclic (e.g. bicyclic) or bridged ring system. Examples of cycloalkyl groups include cyclopropyl, cyclobutyl, cyclopentyl, cyclohexyl and the like. A′ heterocycloalkyl' refers to a cycloalkyl in which one or more of the carbon atoms have been replaced by a heteroatom, such as nitrogen, oxygen or sulphur. ‘Cycloalkeny’ groups correspond to non-aromatic cycloalkyl groups containing at least one carbon-carbon double bond. rheterocycloalkenyr groups correspond to a cycloalkenyl in which one or more of the carbon atoms have been replaced by a heteroatom, such as nitrogen, oxygen or sulphur.

The term ‘alkenyl’ as used herein refers to a monovalent straight- or branched-chain alkyl group containing from 2 to 20 carbon atoms and containing, in addition, at least one carbon-carbon double bond, of either E or Z configuration unless specified. Examples of alkenyl groups include alkenyl groups containing from 2 to 20 carbon atoms (C2 to C20), preferably 2 to 12 atoms (C2 to C12), more preferably 2 to 6 atoms (C2 to C6). Particular examples include alkenyl groups containing 2, 3, 4, 5 or 6 carbon atoms. Examples of alkenyl groups include ethenyl, 2-propenyl, 1-butenyl, 2-butenyl, 3-butenyl, 1-pentenyl, 2-pentenyl, 3-pentenyl, 1-hexenyl, 2-hexenyl, 3-hexenyl and the like.

The term ‘aryl’ as used herein refers to an aromatic carbocyclic ring system containing from 6 to 14 ring carbon atoms. Examples of aryl groups include aryl groups containing from 6 to 10 ring carbon atoms, e.g. 6 ring carbon atoms. An example of an aryl group includes a group that is a monocyclic aromatic ring system or a polycyclic ring system containing two or more rings, at least one of which is aromatic. Examples of aryl groups include aryl groups that comprise from 1 to 6 exocyclic carbon atoms in addition to ring carbon atoms. Examples of aryl groups include aryl groups that are monovalent or polyvalent as appropriate. Examples of monovalent aryl groups include phenyl, benzyl naphthyl, fluorenyl, azulenyl, indenyl, anthryl and the like. An example of a divalent aryl group is 1,4-phenylene. The term rheteroaryr refers to an aromatic carbocyclic ring system as defined for aryl above further comprising one or more heteroatoms, in particular, heteroatoms selected from nitrogen, oxygen and/or sulphur.

As used herein, the term ‘pharmaceutically acceptable salt’ refers to salts prepared from pharmaceutically acceptable non-toxic bases or acids. For example, when the compound of the present invention is acidic, its corresponding salt can be conveniently prepared from pharmaceutically acceptable non-toxic bases, including inorganic bases and organic bases. Examples of such salts include salts of alkali metals such as lithium, sodium, and potassium; salts of alkaline earth metals such as calcium and magnesium; salts of post-transition metal salts such as zinc and aluminium; and salts derived from organic bases such as benzathine, chloroprocaine, choline, tert-butylamine, diethanolamine, ethanolamine, ethyldiamine, meglumine, tromethamine and procaine. Preferred base salts are selected from salts of alkali metals, most preferably sodium. Alternatively, when the compound of the present invention is basic, its corresponding salt can be conveniently prepared from pharmaceutically acceptable non-toxic acids, including inorganic acids and organic acids. Examples of such salts include salts of acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric and p-toluenesulfonic acid and the like. Preferred acid salts are citric, hydrobromic, hydrochloric, maleic, phosphoric, sulfuric, and tartaric acids. In embodiments, the compound may comprise both acid and base salts of basic and acidic moieties of the compound respectively.

Compounds according to the present invention are represented by Formula I or a pharmaceutically acceptable salt thereof:

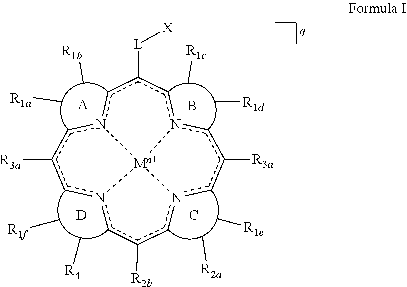

wherein the compound is selected from porphyrins, chlorins, bacteriochlorins or isobacteriochlorins, and wherein:
[symbol] represents a double or single bond;
--- represents a dative or covalent bond between nitrogen and Mⁿ⁺,
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORS, —CSOR₅, —COSR₅, —CSSR₅, —CON(R₅)₂, —CONHR₅, —OR₅, —SRS, —N(R₅)₂, —NHR₅, —CH(OR₅)₂, —CH(OR₅)(SR₅), —CH(SR₅)₂,

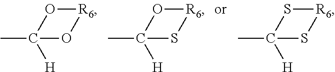

wherein R₅ is monovalent C₁ to C₅-hydrocarbyl or X2, and R₆ is C₁ to C₅ alkylene; or R₂ₐ is taken together with R₁ₑ to form a cyclocarbyl;
R₃ₐ and R_{3b} are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR₇), —CH(SR₇)₂,

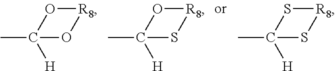

wherein R₇ is monovalent C₁ to C₅-hydrocarbyl or X3, and R₈ to C₁ to C₅-alkylene,
L is a linker;
X, X2 and X3 are fluorophores, which may be the same or different;
Mⁿ⁺ is a cation having a positive charge of integer n, wherein n is from 1 to 3;
q is the overall charge of the compound; and
A, B, C and D each independently represent a five-membered nitrogen-containing heterocycle selected from pyrrole, pyrroline or pyrrolidine rings.

Preferably, the pairs R₁ₐ and R_{1b}, R_{1c} and R_{1d} , R₁ₑ and R₂ₐ, and R_{1f} and R₄ do not form a cyclocarbyl. Therefore, in embodiments R₁ₐ to R_{1f} are independently selected from H or monovalent hydrocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORS, —CSOR₅, —COSR₅, —CSSR₅, —CON(R₅)₂, —CONHR₅, —OR₅, —N(R₅)₂, —NHR₅, —CH(OR₅)₂, —CH(OR₅)(SR₅),

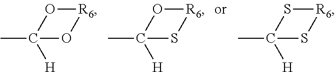

—CH(SR₅)₂, wherein R₅ is monovalent C₁ to C₅-hydrocarbyl or X2, and R₆ is C₁ to C₅ alkylene;
R₃ₐ and R_{3b} are independently selected from H, —CH₃, —CH₂CH₃, —OH, —OCH₃, —OCOCH₃, —SH, —SCH₃, —NH₂, —NHCH₃, or —N(CH₃)₂; and
R₄ is a divalent C₁ to C₁₀-hydrocarbyl-A2 group, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR₇), —CH(SR₇)₂,

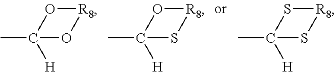

wherein R₇ is monovalent C₁ to C₅-hydrocarbyl or X3, and R₈ is C₁ to C₅-alkylene. The remaining substituents are as defined above.

In Formula I, [symbol] =represents a double or single bond, --- represents a dative or covalent bond between nitrogen and Mⁿ⁺ and A, B, C and D each represent five-membered nitrogen-containing heterocycles selected independently from pyrrole, pyrroline or pyrrolidine rings. As it is stipulated that the compound is selected from porphyrins, chlorins, bacteriochlorins or isobacteriochlorins, the location of the double and single bonds, which of the bonds between nitrogen and Mⁿ⁺ is dative and which covalent, and the identity of rings A to D would be evident to the skilled person.

As described above, porphyrins, chlorins, bacteriochlorins and isobacteriochlorins of Formula I may exist as tautomers. As rings A to D are not identical in Formula I, there will also be multiple isomers of chlorins, bacteriochlorins and isobacteriochlorins, depending on which of the rings is reduced. These isomers may also have tautomers, as discussed above. Formula I includes all of the isomers and tautomers of these molecules, provided that they fall within the definition of porphyrins, chlorins, bacteriochlorins or isobacteriochlorins.

Accordingly, in embodiments in which the compound is selected from porphyrins, the compound may be represented by Formula Ila or I lb or pharmaceutically acceptable salts thereof:

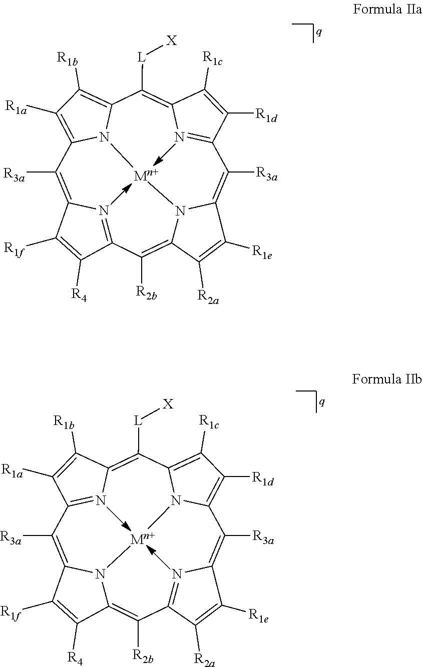

In embodiments in which the compound selected from chlorins, the compound may be represented by any one of Formulas IIla to II Id or pharmaceutically acceptable salts thereof:

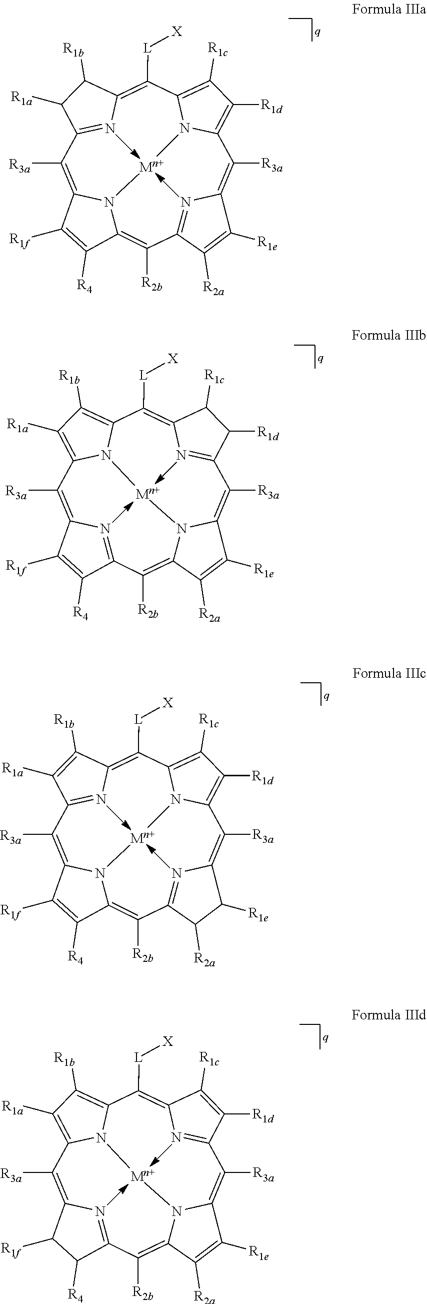

In embodiments in which the compound is selected from bacteriochlorins, the compound may be represented by Formula IVa or IVb or pharmaceutically acceptable salts thereof:

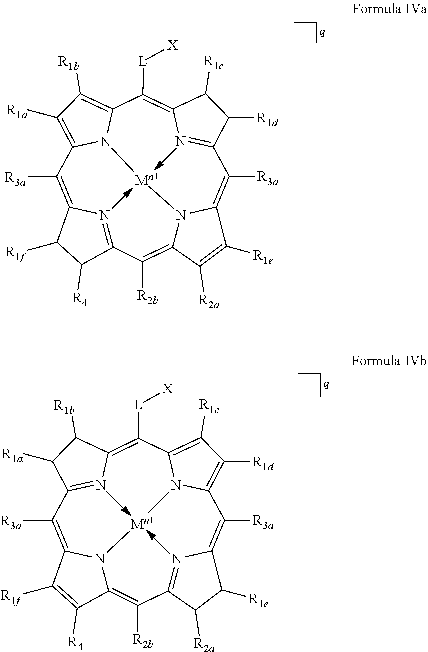

In embodiments in which the compound is selected from isobacteriochlorins, it may be represented by any one of Formulas Va to Vh or pharmaceutically acceptable salts thereof:

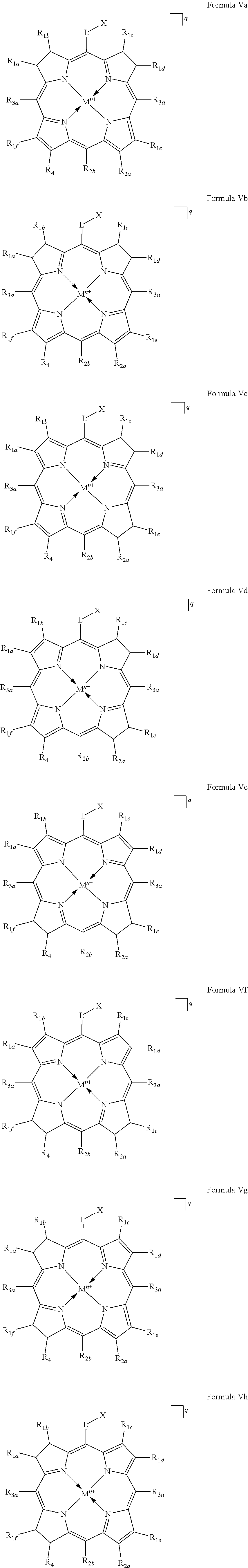

In Formulas IIa to Vh, →represents a dative bond between nitrogen and Mⁿ⁺. Solid bonds between N and Mⁿ⁺ represent covalent bonds (i.e. between an NH which has lost a proton).

The definitions for R₁ₐ to R_{1f}, R₂ₐ, R_{2b}, R₃ₐ, R_{3b}, R₄, L, X, Mⁿ⁺ and q for Formulas Ila, 1lb, IIla to IIId, Iva, IVb and Va to Vh are identical to those defined for Formula I. Formulas IIa, IIb, IIIa to IIId, Iva, IVb and Va to Vh are representations of Formula I. Formulas IIa, IIb, IIIa to IIId, Iva, IVb and Va to Vh encompass resonance structures and are not intended to be limited to only the resonance form shown.

R₁ₐ to R_{1f} may be independently selected from H or monovalent hydrocarbyl, as defined above. Alternatively, one or more of the pairs R₁ₐ and Rib, R_{1c} and R_{1d} , R₁ₑ and R₂ₐ, and R_{1f} and Ra are connected to form a cyclocarbyl. The cyclocarbyl may be selected from a cycloalkyl, heterocyclocalkyl, cycloaklenyl, heterocycloalkenyl, aryl, or heteroaryl. The cyclocarbyl may be a C₄ to C₈ ring, preferably a a C₅ to C₆ ring. The cyclocarbyl may form a polycyclic ring system with the pyrrole, pyrroline or pyrrolidine rings of the tetrapyrrole or reduced tetrapyrrole backbone. The cyclocarbyl may be substituted or unsubstituted.

In embodiments in which R₄ is taken together with R_{1f} to form a cyclocarbyl, this ring is substituted by divalent C₁ to C₁₀-hydrocarbyl-A2 group or by A2 alone, wherein A2 is as defined herein. Preferably, such substitution is at the ring position closest to the group R_{2b} (alternatively, the ring position furthest from R_{3b}).

In preferred embodiments one or more, preferably all, of R₁ₐ to R_{1f}, R₂ₐ, R_{2b} and R₄ are selected from H or monovalent hydrocarbyl.

In embodiments, R₁ₐ to R_{1f} may be independently selected from H or monovalent C₁ to C₆-hydrocarbyl, preferably H, C₁ to C₃-alkyl or C1 to C3 alkenyl, more preferably H, C₁ to C₂-alkyl or C₁ to C₂-alkenyl (namely H, —CH₃, CH₂CH₃ or —CH═CH₂), and most preferably —CH₃.

R₂ₐ and R_{2b} are preferably independently selected from H, monovalent hydrocarbyl or a divalent hydrocarbyl-A1 group. The divalent hydrocarbyl-A1 group is X2 or is a terminating group selected from —COOH, —CSOH, —COSH, —CSSH, —CONH₂, —OH, —SH, —NH₂, —COORS, —CSOR₅, —COSR5, —CSSR₅, —CON(R₅)₂, —CONHR₅, —OR₅, —SRS, —N(R₅)₂, —NHR₅, —CH(OR₅)₂, —CH(OR₅)(SR₅), —CH(SR₅)₂,

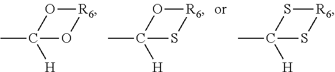

where R₅ is monovalent C₁ to C₅-hydrocarbyl or X2 and R₆ is C₁ to C₅-alkylene. Alternatively, R 2a may be taken together with R₁ₑ to form a carbocycle, as described above. In this embodiment, R_{2b} is still selected from H, monovalent hydrocarbyl group or a monovalent hydrocarbyl-A1 group, as defined above.

In embodiments, R₂ₐ and R_{2b} are independently selected from H, monovalent C₁ to C₁₀-hydrocarbyl group or a divalent C₁ to C₁₀-hydrocarbyl-A1 group. Wherein A1 is X2 or a terminating a group selected from —COOH, —COORS, —CONH₂, —CON(R₅)₂, —OH, —SH, or —NH₂ , wherein R₅ is monovalent C₁ to C₅-hydrocarbyl. In preferred embodiments, R₂ₐ and R_{2b} are independently selected from H, —CH₃, —(CH₂)_{a ′}, —CH₃, —(CH₂)_{a ′}, —COOH, —(CH₂)_{a ′}—COOCH₃ or —(CH₂)_{a ′}, —COOCH₂CH₃ wherein n′ is from 1 to 3, and more preferably R₂ₐ and

R_{2b} are independently selected from —CH₂—CH₂—COOH or —CH₂—CH₂—COOCH3, and most preferably R₂ₐ and R_{2b} are —CH₂—CH₂—COOH. Preferably, R₂ₐ is selected from —CH₂—CH₂—COOH or —CH₂—CH₂—COOCH3, and more preferably R₂ₐ is —CH₂—CH₂—COOH. In this embodiment, R_{2b} may be selected from any of the above, preferably H. In preferred embodiments, R₂ₐ is the same as R₄. In embodiments, R₂ₐ, R_{2b} and R₄ are the same.

R₃ₐ and R_{3b} are independently selected from H, —CH₃, —CH₂CH₃, —OH, —OCH₃, —OCOCH₃, —SH, —SCH₃, —NH₂ , —NHCH₃, or —N(CH₃)₂. It is preferred that R₃ₐ and R_{3b} are small moieties in order to ensure that the compound fits into the binding pocket of HO-1. For this reason, R₃ₐ and R_{3b} are limited to the groups specified herein. As discussed further below, larger groups (e.g. phenyl) at these positions prevents the action of HO-1 on the molecule and the discussed advantages are not observed. In embodiments, R₃ₐ and R_{3b} may independently selected from H, —CH₃, —OH, —SH, or —NH₂ , preferably H.

R₄ is preferably a divalent C₁ to C₁₀-hydrocarbyl-A2 group, wherein A2 is X3 or a terminating group selected —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR7), —CH(SR₇)₂,

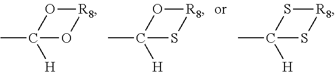

where R₇ is a monovalent C₁ to C₅-hydrocarbyl group or X3, and R₈ is C₁ to C₅-alkylene. Alternatively, R₄ may be taken together with R_{1f} to form a cyclocarbyl as defined above, which is substituted by a C₁ to C₁₀-monovalent hydrocarbyl-A2 group or by A2 alone, wherein A2 is as defined above. Preferably, the cyclocarbyl is substituted by a C₁ to C₃-monovalent hydrocarbyl-A2 group or by A2 alone.

In embodiments, R₄ may be a C₁ to C₅-alkyl-A2 group, C₁ to C₅-ether-A2 group or C₁ to C₅-thioether-A2 group and A2 is X3 or selected from —COOH, —COOR₇, —CONH₂, —CON(R₅)₂, —OH, —SH, or —NH₂ , wherein R₇ is a monovalent C₁ to C₂ -hydrocarbyl group. Preferably R₄ is selected from —(CH₂)_{n″}—COOH or —(CH₂)_{n″}—COOCH₃ wherein n″ is from 1 to 3, more preferably —(CH₂)₂ —COOH (propanoic acid) or —(CH₂)₂ —COOCH₃ (propanoic acid methyl ester), most preferably —(CH₂)₂ —COOH (propanoic acid). In embodiments, R₂ₐ and R₄ can be independently selected from —(CH₂)2—COOH or —(CH₂)2—COOCH3, most preferably both are —(CH₂)₂ —COOH.

Thus, in preferred embodiments, the compound according to the present invention is of Formula Ia or a pharmaceutically acceptable salt thereof:

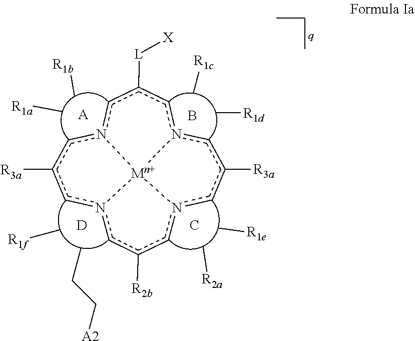

wherein R₄ of Formula I is —(CH₂)₂-A2. A2 and the other substituents are as described herein. Preferably, A2 is selected from —COOH, —COOR₇, -OH or -SH wherein R₇ is a C1 to C2 monovalent hydrocarbyl group. More preferably, A2 is —COOH or —COOCH₃, most preferably —COOH.

In embodiments wherein the compound of Formula la is a porphyrin, the structure is based on the structure of di-methyl-deutero-heme (DMD). DMD is an analogue of heme (PPIX), which is the endogenous substance degraded by HO-1, and is known to display an activity towards HO-1.

In embodiments, A1 does not comprise or consist of a fluorophore (X2) and A2 does comprise or consist of a fluorophore (X3). In embodiments, A2 does not comprise or consist of a fluorophore (X3) and A1 does comprise or consist of a fluorophore (X2). In embodiments, neither of A1 or A2 comprise or consist of a fluorophore (X2 or X3). In embodiments, both of A1 and A2 comprise or consist of a fluorophore (X2 and X3).

As discussed above, wherein either or both of A1 and/or A2 comprise or consist of a fluorophore (X2 and/or X3), the fluorophore may be any suitable fluorophore, and may be selected from the fluorophores discussed below with respect to X. Preferably, the inclusion of fluorophores X2 and/or X3 does not negatively impact the solubility of the compound.

As will be appreciated, wherein either A1 or A2 comprise or consist of a fluorophore (X2 or X3), or wherein A1 and A2 each comprise or consist of a different fluorophore (X2 and X3), synthesis may involve attaching the fluorophore or fluorophores independently, using protecting group strategy.

In embodiments, fluorophores X, X2 and X3 have excitation and/or emission maxima at different wavelengths. In embodiments, ratiometric analysis of the emission intensity of the fluorophores may be performed.

L is a linker which connects the 5 position of the tetrapyrrole or reduced tetrapyrrole backbone of the compound to fluorophore X. The identity of L is not particularly limited. In embodiments, L may represent a direct bond such that fluorophore X may be connected directly to the 5 position of the tetrapyrrole or reduced tetrapyrrole backbone. In alternative embodiments, L may be a divalent C₁ to C₂₀-hydrocarbyl, preferably a C₁ to C₂₀-alkylene, C₁ to C₂₀-ether, C₁ to C₂₀-aryl or C₁ to C₂₀-heteroaryl, more preferably a C₁ to C₂₀-alkylene, C₁ to C₂₀-aryl or C₁ to C₂₀-heteroaryl comprising 1,4-phenylene and/or 1,2,3-triazole.

A 1,2,3-triazole may be formed by a ‘click’ reaction between an alkyne and an azide. This reaction may be performed between a tetrapyrrole or reduced tetrapyrrole backbone which has been modified to include an azide or an alkyne and fluorophore X which has been modified to include the other of an azide or an alkyne in order to synthesise a compound of Formula I.

In embodiments, L may be

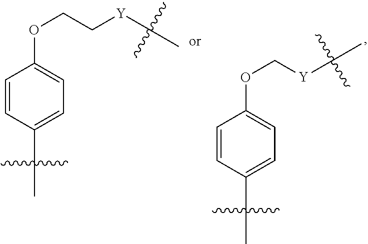

wherein Y is selected from O, NR₉ or 1,2,3-triazole, wherein R₉ is H or C₁ to C6-hydrocarbyl.

X is a fluorophore and may be an aromatic or heteroaromatic compound optionally selected from a pyrene, anthracene, naphthalene, acridine, stilbene, indole, benzindole, oxazole, thiazole, thiazine, benzothiazole, cyanine, carbocyanine, salicylate, anthranilate, coumarin, fluorescein and/or rhodamine and derivatives thereof. Preferably, X is selected from coumarin, fluorescein and derivatives thereof, indocyanine green and methylene blue and derivatives thereof.

In embodiments, it is preferable to select a fluorophore which absorbs and/or emits in the near IR (NIR) window, where light has its maximum depth of penetration in tissue. The NIR window is defined as a wavelength of from 650 nm to 1350 nm. An example of a NIR dye is indocyanine green (ICG), which has been tested in human subjects and is extensively used in fluorescence angiography, further examples of NIR dyes include methylene blue, aza-BODIPY, AlexaFluor 647, Cy7 Cy7.5, Fluorescein, and Alexa 488. This allows for in vivo and ex vivo imaging at greater depths.

In embodiments, fluorescein and analogues thereof are preferred, as their emission and excitation wavelengths constitute default wavelengths for most equipment. This makes implementation more facile. This is particularly true in imaging experiments which are not in vivo, such as ex vivo and in vitro and when the compounds are used as a research reagent.

In embodiments, X is methylene blue. This is licensed to treat patients and clinically used at high concentration to treat methemoglobinemia. It has a minimal and well-characterised toxicity profile in human subjects.

Mⁿ⁺ may be any cation which can coordinate to the porphyrin, chlorin, bacteriochlorin or isobacteriochlorin, such as a metal cation or an organic cation. Preferably Mⁿ⁺ is not toxic once bound. The value of n is from 1 to 3, preferably from 1 to 2. In embodiments, Mⁿ⁺ may be ammonium or a metal cation, preferably ammonium or an iron, zinc or magnesium cation (e.g. NH₄⁺, Fe²⁺, Fe³⁺, Zn²⁺ or Mn²⁺), more preferably an iron cation, most preferably an iron cation selected from iron(II) and iron(III).

The charge q of the molecule will depend on the charge of Mⁿ⁺ and the charge of any of the substituents on the tetrapyrrole or reduced tetrapyrrole backbone, namely the charge of R₁ₐ to R_{1f}, R₂ₐ, R_{2b}, R₃ₐ, R_{3b}, R₄, L and X. As discussed above, the tetrapyrrole or reduced tetrapyrrole backbone will have a formal charge of 2− due to the loss of the two protons on the saturated nitrogen atoms which form a covalent bond to Mⁿ⁺. Preferably, q will be between 3− and 3+, more preferably between 2− and 2+ and most preferably between 1− and 1+. The compound may be neutral, and thus q may be 0. In embodiments, a compound having a M⁺ cation (i.e. n=1) may have a charge q of 1−. In embodiments, a compound having a M²⁺ cation (i.e. n=2) may have a charge q of 0. In embodiments, a compound having a M³⁺ cation (i.e. n=3) may have a charge q of 1+.

The charge q′ of compounds of Formula Vla to Vlc may be between 3− and 3+, more preferably between 2− and 2+ and most preferably between 1− and 1+. For Formula Vla, q′ may be as described above for q for different Mⁿ⁺ cations. Preferably, Formulas Vlb and Vlc have a q′ of 0.

The charge q″ of [Z-X]^{q″} may be between 3− and 3+, more preferably between 2− and 2+ and most preferably between 1− and 1+. In preferred embodiments, q″ is 0.

In embodiments in which q, q′ and/or q″ are not 0, the compounds may be associated with a counterion. Such a complex will be a pharmaceutically acceptable salt of the compound, as defined herein. Any suitable counterion may be used, the identity of which will depend on whether the charge on the compound is positive (and thus requires a negative counterion) or negative (and thus requires a positive counterion). In embodiments, the counterion may be a halide (X⁻), such as chloride (Cl⁻), or hexafluorophosphate (PF₆⁻), as these may aid with solubility of the compound.

Fluorescence Resonance Energy Transfer (FRET), also known as FOrster Resonance Energy Transfer, describes energy transfer between two chromophores. FRET relies on the distance-dependent transfer of energy from a donor molecule to an acceptor molecule through non-radiative dipole—dipole coupling. A pair of molecules that interact in such a manner are often referred to as a donor/acceptor pair. The donor molecule is the chromophore that initially absorbs the energy and the acceptor is the chromophore to which the energy is subsequently transferred. When the donor and acceptor chromophores are different fluorophores, FRET can be detected by the appearance of fluorescence of the acceptor or by quenching of donor fluorescence.

The FRET efficiency depends on the spectral overlap of the donor emission spectrum and the acceptor absorption spectrum, with better overlap leading to improved FRET efficiency. The efficiency of FRET energy transfer is also inversely proportional to the sixth power of the distance between donor and acceptor, making FRET extremely sensitive to small changes in distance. Due to its sensitivity to distance, FRET can be used to investigate molecular interactions.

In preferred embodiments, X and the tetrapyrrole or reduced tetrapyrrole backbone of the porphyrins, chlorins, bacteriochlorins or isobacteriochlorins represent a FRET pair. In order for these moieties to represent a FRET pair they must be in close proximity to one another (typically 10-100 Å), which is achieved in the compounds of the present invention. In addition, the absorption or excitation spectrum of the acceptor must overlap the fluorescence emission spectrum of the donor. Thus, preferred fluorophores depend upon the identity of the tetrapyrrole or reduced tetrapyrrole backbone, as each of these have their own spectral characteristics. The pairing of tetrapyrrole or reduced tetrapyrrole backbones and fluorophores with overlapping spectral characteristics may be readily achieved with the benefit of the present disclosure and based on routine measurements of the emission and excitation spectra of the donor and accepter and verifying overlap (e.g. using ThermoFisher spectral viewer) or through the use of known reference standards (tables of known FRET pairs can be found in e.g. the ThermoFisher Molecular Probes Handbook, Chapter 1). Either of the tetrapyrrole or reduced tetrapyrrole backbones and the fluorophore may be the donor and the other the acceptor, provided that the relevant excitation and emission spectra overlap. Thus, fluorescence from X may be quenched in the compound according to the present invention following excitation of X due to FRET transfer to the tetrapyrrole or reduced tetrapyrrole backbone. Alternatively, fluorescence from X may be observed in the compound according to the present invention following excitation of the tetrapyrrole or reduced tetrapyrrole backbone due to FRET transfer from the tetrapyrrole or reduced tetrapyrrole backbone to X.

In embodiments, Xis preferably coumarin or fluorescein or a derivative thereof when the compound is a porphyrin. In other embodiments, the compound is a chlorin, bacteriochlorin or isobacteriochlorin and X is indocyanine green or methylene blue or a derivative thereof. In both of these embodiments, X is the donor and the tetrapyrrole or reduced tetrapyrrole backbone of the porphyrin, chlorin, bacteriochlorin or isobacteriochlorin is the acceptor. Thus, fluorescence of X is quenched following excitation of X due to the FRET interaction with the tetrapyrrole or reduced tetrapyrrole acceptor.

In embodiments, the molecule is a porphyrin and X is indocyanine green or methylene blue or a derivative thereof. In this embodiment, X is the acceptor and the tetrapyrrole backbone of the porphyrin is the donor.

Upon reaction of HO-1 with the compound according to the present invention, the tetrapyrrole or reduced tetrapyrrole backbone is believed by the inventors to be de-cyclised at the 5 position. Following this reaction, carbon atom 5 remains connected to L-X, whilst the remaining molecule becomes a linear tetrapyrrole or reduced tetrapyrrole. As X is no longer bound to the tetrapyrrole or reduced tetrapyrrole backbone, these moieties are no longer maintained in close proximity. Thus, FRET between these moieties no longer occurs. Thus, fluorescence from X is modulated following de-cyclisation of the compound by HO-1. Due to this, these FRET probes provide a large signal to noise ratio, resulting in sensitive imaging of HO-1 activity.

Whether fluorescence modulation results in an increase or a decrease in fluorescence from X depends on whether X is the donor or acceptor in the FRET pair. In embodiments in which X is the donor, fluorescence from X is quenched in compounds according to the present invention, resulting in increased fluorescence following the action of HO-1. Thus, the observation of fluorescence from X identifies HO-1 activity.

Conversely, in embodiments in which X is the acceptor, fluorescence from X is observed when the tetrapyrrone or reduced tetrapyrrole backbone is excited. Thus, the reduction of fluorescence from X identifies HO-1 activity. As will be appreciated, wherein X2 and/or X3 are additionally present, these fluorophores may additionally be involved in FRET pairs. It has been found by the inventors that other molecules including a fluorophore at the 5-position of a tetrapyrrole backbone would not be de-cyclised by HO-1 due to the presence and/or absence of substituents around the tetrapyrrole backbone. In particular, large moieties, such as phenyl, at the 10, 15 and 20 position (i.e. occupied by R₃ₐ R_{2b} and R_{3b} in the compound according to the present invention) has been found to prevent HO-1 acting on the compound. In addition, it is believed that the presence of a carboxylic acid or methyl ester, in particular a carboxylic acid, terminating the R₄ hydrocarbyl group improves the activity of HO-1 on the compound. This is particularly true when the R₄ chain is a C3 chain and when R₂ₐ is propanoic acid. Thus, R₄ is preferably a propanoic acid group or a propanoic acid methyl ester, preferably propanoic acid, and R₂ₐ is preferably propanoic acid.

Having regard to the crystal structure of HO-1, these effects are believed to be due to the size and shape of the binding pocket of HO-1. The binding of heme in the active site of HO-1 is widely known (PDB accession 1N45, PMID: 12500973). The crystal structure of HO-1 is described in literature, for example in Lad, L et al., J. Biol. Chem., 2003, 278, 7834-7843.

Inspection of heme in the binding pocket of HO-1 reveals that there is a relatively large pocket at a deep interior face of the active site. This corresponds with expected location of fluorophore X at the 5 position. It also demonstrates little additional space between the external border of heme and internal border of active site at positions 10 and 15 of heme. It also appears that the propinoic acid is located in close proximity with positively charged Lys and Arg side chains in the HO-1 active site, implying an electrostatic bond between heme propanoate O^{δ-}/O⁻ on the carboxylic acid moiety and positively charged side chains of Lys and Arg.

Without being bound by theory, it is believed that R₄ interacts with positively charged residues in the HO-1 binding pocket in a similar fashion, in which interactions are maximised when R₄ is 3 atoms long and terminated in a carboxylic acid (e.g. R₄ is propionic acid), as in heme. This results in improved break-down of the compound, and thus a greater fluorescent response observed. This is also believed true of R₂ₐ, which is preferably as described above for R₄. The advantageous binding ability of substrates having propionates at both the R₄ and R₂ₐ positions is discussed in Peng, D et al., Biochemistry, 2012, 51, 36, 7054-7063 with regard to HO-1. Peng, D et al further demonstrates that substitution such as a propionate is well tolerated at the R_{1f} position. The relevance of propionate mediated salt bridge interactions with HO-1 has been further demonstrated in Wang, J et al., Biochemistry, 2006, 45, 1, 61-73. Wang et a/ also show that mutating an arginine and lysine in the HO-1 binding pocket, thought to be responsible for binding to the propionates, significantly decreases regioselectivity. This suggests that the propionates can play a role in anchoring heme in the binding pocked of HO-1 for regioselective alpha-cleavage.

In addition, it is believed that large substituents in the R₃ₐ and R_{3b} positions, such as phenyl, prevent binding of HO-1 as the binding pocket is not wide enough to accommodate such groups.

Due to the action of HO-1 on the compounds according to the present invention, the compounds can be used to image HO-1 and diagnose diseases which are characterised by HO-1 over-expression, as the compounds will be metabolised by this enzyme. Therefore, the detection of fluorescence and/or fluorescence modulation can be used to identify and locate increased levels of HO-1. This is particularly advantageous in embodiments in which the molecule includes a FRET pair, as described herein, where the action of HO-1 removes the fluorophore from the tetrapyrrole or reduced tetrapyrrole backbone, preventing FRET and leading to fluorescence modulation.

Diseases which are characterised by HO-1 over expression include intraplaque haemorrhage, intracerebral haemorrhage, intracranial haemorrhage, subarachnoid haemorrhage, ruptured aortic aneurysm (atherosclerotic and non-atherosclerotic), neurodegenerative disease (also involves microhaemorrhage), retinal haemorrhage, intravascular haemolysis, red cell lysis related to thrombosis (including in COVID-19), tissue haemorrhage in other sites, acute coronary syndrome and/or stroke caused by intraplaque haemorrhage and/or atherosclerosis.

Atherosclerosis is a condition where arteries become clogged by plaques, formed from fat, cholesterol, calcium, and other substances found in the blood. These plaques cause the arteries to harden and narrow, restricting the blood flow and oxygen supply to vital organs, and increasing the risk of blood clots that could potentially block the flow of blood to the heart or brain.

Atherosclerosis does not typically display any symptoms initially, however it can eventually cause life-threatening problems. In some instances, intraplaque haemorrhage can occur, where vulnerable plaque coating the artery walls ruptures. This can lead to blood clots, which themselves can cause the onset of ischemic stroke and acute coronary syndrome. Additionally, intraplaque haemorrhage can lead to the development of more advanced vulnerable plaques which can lead to the development of further blood clots. Thus, early detection of vulnerable plaques may prevent further problems from developing.

In atherosclerosis, HO-1 is induced during intraplaque haemorrhage. Thus, increased levels of HO-1 can be used to diagnose such a haemorrhage. In addition, plaque expression of HO-1 can be used to predict plaque rupture. Without being bound by theory, this is believed to be because intraplaque haemorrhage promotes plaque vulnerability, whilst HO-1 induction reduces plaque vulnerability. Thus, the detection of increased levels of HO-1 can indicate plaque vulnerability before plaque rupture, allowing for treatment prior to rupture. This allows for plaque to be prophylactically treated with percutaneous coronary intervention (PCI) such as angioplasty and stenting, in addition to enhanced preventive treatments such as anticoagulants, antiplatelets, antihypertensives, lipid-lowering agents, hypoglycaemic agents as indicated.

Acute coronary syndrome is defined as the formation of a blood clot inside a blood vessel of the heart. This blood clot may restrict blood flow within the heart, leading to heart tissue damage, or a myocardial infarction (heart attack). As intraplaque haemorrhage can result in blood clots, depending upon the location of the plaque it can result in acute coronary syndrome. Thus, HO-1 can be used to diagnose whether a heart attack is the result of acute coronary syndrome.

A stroke occurs when the blood supply to part of the brain is cut off. Thus, the sooner a person receives treatment for a stroke, the less damage is likely to happen. As intraplaque haemorrhage can result in blood clots, depending upon the location of the plaque it can result in a stroke.

There are multiple types of stroke, including ischemic stroke and hemorrhagic stroke. The compounds of the present invention can be used to differentiate between these types of stroke, as stroke caused by intraplaque haemorrhage (ischemic stroke) results in increased HO-1 levels. This is particularly important in this instance, because whilst anticoagulants or thrombolytics can improve ischemic stroke, they can worsen hemorrhagic stroke. Thus, it is important to understand the cause of the stroke before administering these treatments.

Diagnosis can be in vivo, ex vivo, or in vitro.

In embodiments, diagnosis is preferably performed in vivo as this provides more accurate details regarding the location of HO-1 in the subject, which may aid with subsequent treatment. In some instances, in vivo diagnosis may also be less invasive. For example, in vivo diagnosis may take place with only one modality as fewer different catheters need be inserted into the patient, allowing for a quicker and less disruptive procedure. For this reason, treatment may also be advantageously provided in the same procedure, rather than in a subsequent procedure. Point of care diagnosis on peripheral blood would be preferable as it would avoid any catheter insertion.

Ex vivo and in vitro diagnosis also presents particular benefits. For example, blood may be taken from a subject and rapidly tested for HO-1 levels, with minimal equipment being necessary for such an assessment. Thus, the compounds according to the present invention may be used in the diagnosis of diseases in situations where minimal equipment is available, for example at point of care in emergency situations e.g. by paramedics. This information may then be used to advise what treatment should be used, without waiting to arrive at a location with more advanced equipment e.g. a hospital. This allows for more rapid and/or more appropriate treatment to be administered. Moreover, the removal of blood to be tested requires minimal interference with the subject, reducing the need for imaging of the subject themselves. This may reduce subject contact time and subject stress. It also does not require the injection of the composition into the body.

This is particularly useful in the diagnosis of stroke. Typically, such diagnosis requires a CT-scan of the head, which itself requires the use specialist equipment which is only available at specific locations. As discussed above, it is important to understand the cause of the stroke prior to the use of certain treatments. The compounds of the present invention can be used to differentiate between these types of stroke by testing the patient's blood, as stroke caused by intraplaque haemorrhage results in increased HO-1 levels. Thus, the cause of the stroke can be determined quickly without the need for a CT-scan, allowing for more rapid administration of the appropriate treatment. As the speed of treatment is crucial in limiting the damage caused by a stroke, such testing provides clear advantages.

Thus, the compounds according to the present invention may be used in a method of diagnosis, optionally of a disease characterised by HO-1 over expression. In particular, they may be used in a method of diagnosis in vivo of intraplaque haemorrhage, acute coronary syndrome and/or stroke caused by intraplaque haemorrhage and/or atherosclerosis.

The compounds according to the present invention may be used in a method for in vitro and/or ex vivo diagnosis of a disease characterised by HO-1 over expression. In particular, they may be used in a method for in vitro and/or ex vivo diagnosis of intraplaque haemorrhage, acute coronary syndrome and/or stroke caused by intraplaque haemorrhage and/or atherosclerosis.

The method of diagnosis in vivo preferably comprises administering the compound to a subject. In embodiments in which acute coronary syndrome is being diagnosed, the compound is preferably administered into the coronary artery. The method of diagnosis in vitro and/or ex vivo may comprise contacting the compound with biological matter removed from the subject, such as blood or tissue, preferably blood.

The method of in vivo, in vitro and/or ex vivo diagnosis may include exciting the fluorophore and recording the fluorescent response. In embodiments, the method may further include providing light at a wavelength within the absorbance spectrum of X or the tetrapyrrole or reduced tetrapyrrole backbone of the porphyrins, chlorins, bacteriochlorins or isobacteriochlorins and detecting light emitted by X. In this method, the detection of fluorescence modulation identifies the presence of HO-1.

Thus, the compound according to the present invention may be used in the in vitro and/or ex vivo diagnosis of intraplaque haemorrhage, intracerebral haemorrhage, intracranial haemorrhage, subarachnoid haemorrhage, ruptured aortic aneurysm (atherosclerotic and non-atherosclerotic), neurodegenerative disease (also involves microhaemorrhage), retinal haemorrhage, intravascular haemolysis, red cell lysis related to thrombosis (including in COVID-19), tissue haemorrhage in other sites, acute coronary syndrome and/or stroke caused by intraplaque haemorrhage and/or atherosclerosis.

The benefits of in vivo detection are facilitation of immediate minimally invasive, detection of HO-1 and thus diagnosis of disease. This reduces trauma to a patient, and informs decisions on treatment type and location and facilitate immediate treatment by minimally invasive surgical procedures such as angioplasty and stenting. Surgical interventions may also be done in the same procedure as the in vivo detection.

Such in vitro and ex vivo use will be beneficial in instances where access to imaging equipment suitable for subjects is not available. For example, it may be used for determination of whether a patient has experienced a stroke at first instance, before being transferred to a hospital for further imaging and treatment. Such a test could be similar to a diabetes test, requiring only a small amount of blood. This will enable suitable treatments to be determined immediately, without waiting to reach a hospital or other facility. Thus, the compounds of the present invention can be used at the point-of-care. The compounds may be included in a point-of-care device.

As the compounds according to the present invention can identify HO-1 over expression, they may be used as part of a method of treatment of a disease which is characterised by such over-expression, where the method of treatment requires determination of the location for treatment. Thus, the compounds according to the present invention may be used in a method of treatment of a disease characterised by HO-1 over expression, such as acute coronary syndrome and/or stroke caused by intraplaque haemorrhage and/or atherosclerosis.

The method may include exciting the fluorophore and recording the fluorescent response. In embodiments, the method of treatment may comprise administering the compound to a subject, providing light at a wavelength within the absorbance spectrum of X or the tetrapyrrole or reduced tetrapyrrole backbone of the porphyrins, chlorins, bacteriochlorins or isobacteriochlorins, detecting light emitted by X and treating the location in the subject in which fluorescence modulation is observed.

The compounds according to the present invention may be used in a method of imaging heme oxygenase 1 and/or intraplaque haemorrhage. Such methods may comprise contacting the compound with a sample. The method of imaging may include exciting the fluorophore and recording the fluorescent response. In embodiments, the method may further include providing light at a wavelength within the absorbance spectrum of X or the tetrapyrrole or reduced tetrapyrrole backbone of the porphyrins, chlorins, bacteriochlorins or isobacteriochlorins and detecting light emitted by X. In this method, the detection of fluorescence modulation identifies the presence of HO-1.

Thus, the compounds according to the present invention may be used as contrast agent for imaging HO-1 and/or intraplaque haemorrhage.

In addition, the compounds according to the present invention may be used as a research reagent, preferably for the detection of HO-1.

There is currently no single laboratory reagent that would allow for robust and convenient detection of HO-1 enzyme activity. There are a number of kits that detect HO-1 protein (by ELISA) but this is not the same as probing for active enzyme. Such use will thus be beneficial in the development of, for example, treatments and other therapeutic agents which are intended to treat or prevent diseases which are characterised by increased HO-1 activity, for example, Cardiovascular disease, stroke, neurodegenerative disease, lung disease, haemorrhage, viral infections.

As used herein, the term “treatment” or “treating” refers to an amelioration of a disease or disorder, or at least one discernible symptom thereof. The term “treatment” or “treating” refers to inhibiting or reducing the progression of a disease or disorder, either physically, e.g., stabilization of a discernible symptom, or physiologically, e.g., stabilization of a physical parameter, or both.

The compounds may be provided in any suitable form, for example an aqueous or oil solution or suspension, dispersible powder or granule or emulsion. The compounds may be included as part of a formulation. The term “formulation” is used herein to describe the combination of the compounds with one or more vehicles, such as pharmaceutically acceptable vehicles.

As used herein, the term “pharmaceutically acceptable” means approved by a regulatory agency such as that of the Federal or a state government or listed in the U.S. Pharmacopeia or other generally recognized pharmacopeia for use in humans. The term “vehicle” refers to a diluent, adjuvant, excipient, or carrier with which a composition according to the present invention is administered. Such pharmaceutical vehicles can be liquids, such as water and oils, including those of animal, vegetable or synthetic origin, such as flaxseed oil, peanut oil, soybean oil, mineral oil, sesame oil, fish oil and the like, preferably being flaxseed oil and/or fish oil. The pharmaceutical vehicles can be saline, gum acacia, gelatin, starch paste, talc, keratin, colloidal silica, urea, and the like. The vehicle may also be liposome based, as described hereinbefore. In addition, auxiliary, stabilizing, thickening, lubricating and colouring agents may be used. When administered to a subject, the compositions of the embodiments and pharmaceutically acceptable vehicles are preferably sterile. Saline solutions and aqueous dextrose and glycerol solutions can also be employed as liquid vehicles. Suitable pharmaceutical vehicles also include excipients such as inert diluents such as calcium carbonate, sodium carbonate, lactose, calcium phosphate or sodium phosphate; granulating and disintegrating agents, such as corn starch or alginic acid; binding agents such as starch, gelatin or acacia; lubricating agents such as magnesium stearate, stearic acid or talc; and/or glucose, lactose, sucrose, gelatin, glycerol, propylene, glycol, water, ethanol and the like. The formulation, if desired, can also contain minor amounts of wetting or emulsifying agents, or pH buffering agents. The formulation may also contain nonpyrogenic emulsion for intravenous injection/infusion.

Vehicles are compatible with the other ingredients of the formulation and do not interfere with the activity of the probe. Thus, the vehicle preferably does not adversely affect tissue samples or cells to which is it administered. Pharmaceutically acceptable vehicles are compatible with the other ingredients of the formulation as well as non-injurious to the subject.

In embodiments, the vehicle includes one or more liquid vehicles. The liquid vehicle may be a pharmaceutically acceptable liquid vehicle. A pharmaceutically acceptable liquid vehicle is preferably used in embodiments in which the compounds are administered in vivo, in particular when the compounds are administered intravenously. In embodiments where the compounds are used in vitro, ex vivo, or as a research reagent, the liquid vehicle may be any suitable liquid vehicle and need not be a pharmaceutically acceptable liquid vehicle.

Preferably, the formulation comprises the composition and a liquid pharmaceutical vehicle comprising water. In embodiments, the liquid pharmaceutical vehicle may be aqueous. Thus, the formulation may comprise the composition and an aqueous liquid pharmaceutically acceptable vehicle.

In embodiments, the formulation is suitable for in vivo administration.

The compounds according to the present invention may be administered to the subject in any suitable form. Preferably, for in vivo diagnosis, imaging and/or treatment, the compounds may be administered by intravenous injection, preferably the compounds are administered by direct intracoronary injection.

In embodiments where administration as intravenous administration is envisaged, the composition may be in the form of a sterile injectable aqueous or oleaginous suspension. This suspension can be formulated according to methods well known in the art using suitable dispersing or wetting agents and suspending agents. In embodiments where administration as intravenous administration is envisaged, the composition may be in the form of a sterile injectable emulsion. This emulsion can be formulated according to methods well known in the art using suitable emulsifying agents. The sterile injectable preparation can also be a sterile injectable solution, emulsion or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils can be employed conventionally as a solvent or suspending medium. For this purpose, any bland fixed oil can be employed including synthetic mono or diglycerides. In addition, fatty acids such as oleic acid can likewise be used in the preparation of injectable preparations.

In in vitro, ex vivo use and use as a research reagent, the compounds according to the present invention may be in any suitable form. For example, the compounds may be dissolved in a liquid vehicle which need not be a pharmaceutically acceptable liquid vehicle, such as chloroform.

In in vitro, ex vivo use and use as a research reagent, the compound or formulation may be administered directly to a sample or to a compound/ composition of interest.

The subject may be a vertebrate, preferably a mammal, more preferably a human. The subject may be an adult or a child.

Further provided is a modified tetrapyrrole or reduced backbone which can be easily functionalised with various fluorophores selectively at the 5 position. This allows for easy modification of the tetrapyrrole or reduced tetrapyrrole backbone at the desired position and allows for the facile synthesis of a library of compounds according to the present invention. This avoids the need for lengthy synthesis of each probe individually.

The modified tetrapyrrole or reduced tetrapyrrole backbone comprises an alkyne or azide functional group at the 5 position. This allows for reaction with a fluorophore functionalised with the other of an azide or alkyne. These molecules can be reacted together using the well-characterised covalent cycloaddition reaction between the azide and the alkyne.

Thus, there is also provided a method of synthesising a compound as disclosed herein, the method comprising reacting a compound of Formula Vla to Vlc or a pharmaceutically acceptable salt thereof:

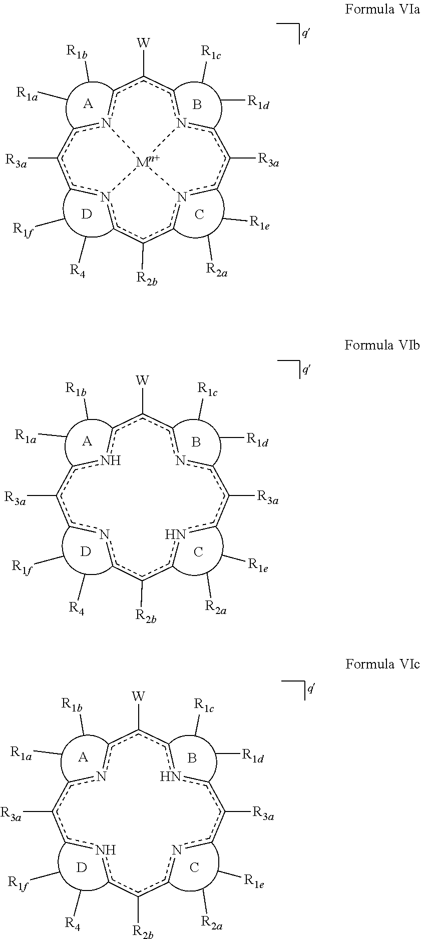

wherein the compound is a porphyrin, chlorin, bacteriochlorin or isobacteriochlorin, and wherein:
[symbol] represents a double or single bond;
[symbol] represents a dative or covalent bond between nitrogen and Mⁿ⁺,
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORS, —CSOR₅, —COSR₅, —CSSRs, —CON(Rs)₂, —CONHR₅, —OR₅, —N(Rs)₂, —NHR₅, —CH(ORs)₂, —CH(OR₅)(SR₅), —CH(SR₅)₂,

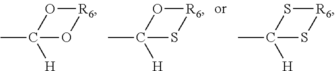

wherein R₅ is monovalent C₁ to C₅-hydrocarbyl or X2, and R₆ is C₁ to C₅ alkylene; or R₂ₐ is taken together with R₁ₑ to form a cyclocarbyl;
R₃ₐ and R_{3b} are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR₇), —CH(SR₇)₂,

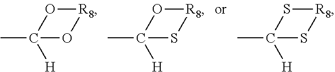

wherein R₇ is monovalent C₁ to C₅-hydrocarbyl or X3, and R₈ to C₁ to C₅-alkylene;
L is a linker;
X2 and X3 are fluorophores which may be the same or different;
Mⁿ⁺ is a cation having a positive charge of integer n, wherein n is from 1 to 3;
q′ is the overall charge of the compound;
A, B, C and D each independently represent a five-membered nitrogen-containing heterocycle selected from pyrrole, pyrroline or pyrrolidine rings; and
W comprises an azide or an alkyne functional group,

with a compound having the formula [Z-X]^{q″}, wherein X is a fluorophore which may be the same as or different from X2 and X3, q″ is the overall charge of the compound, and Z comprises an azide or an alkyne functional group,

wherein one of W and Z comprises an azide functional group and the other comprises an alkyne functional group.

In embodiments, R₁ₐ to R_{1f}, R₂ₐ, R_{2b}, R₃ₐ, Rab, R₄, L, Mⁿ⁺, A, B, C and D may be as described hereinabove.

Preferably, the azide or alkyne functional group of W is not directly attached to the tetrapyrrole backbone. In embodiments, W further comprises a linker. Preferably ,the azide or alkyne functional group is attached to a linker and the linker is attached to the tetrapyrrole backbone. In embodiments, W comprises divalent C₁ to C₂₀-hydrocarbyl terminated with an azide or an alkyne. Preferably, the hydrocarbyl comprises a phenyl. In embodiments, W is

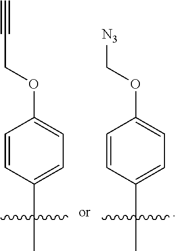

In embodiments, Z is either an azide or an alkyne.

The reaction between azide and alkyne is a covalent cycloaddition reaction. Preferably the reaction is a click reaction. The reaction may be a copper-catalysed azide-alkyne cycloaddition (CuAAC), more details on click reactions can be found in Liang, L, et al., Coord, 2011, 225, 23-24, 2933-2945. Thus, the reaction may be performed in the presence of a copper-containing catalyst, preferably a catalyst comprising ascorbate and copper(I) and/or copper(II). The reaction may be a strain-promoted azide-alkyne cycloaddition (SPAAC). Thus, the reaction may be performed without the presence of copper. This can be beneficial as it eliminates the possibility of cytotoxic copper remaining in the final product. In such a reaction, the alkyne is activated by strain. For example, the alkyne may a be cyclooctyne, such as difluorooctyne, dibenzylcyclooctyne, or biarylazacyclooctynone.

The present invention is further described by way of the following Examples, which are provided for illustrative purposes and are not in any way intended to limit the scope of the invention as claimed.

### Examples

### Example 1—Probe Synthesis

Various porphyrin-coumarin probes were synthesised to demonstrate the effectiveness of probes according to the present invention. These probes are shown in Table 6:

7-Hydroxymethyl-coumarin and 7-aminomethyl-coumarin derivatives were selected as the donor fluorophores due to their excellent spectral overlap with the absorbance spectrum of porphyrins. Di-methyl-deutero-heme (DMD) was selected as the porphyrin backbone for the synthesis of compounds according to the present invention as opposed to ‘free’ heme (PPIX) as it allows for more facile functionalisation of the 5 position selectively, whilst also being a substrate for HO-1. Comparative examples were based on 5-(4′hydroxyphenyl)-porphyrin (HP-TPP), which includes a phenol group at the 5 position and phenyl groups at positions 10, 15 and 20.

In these compounds, the coumarin fluorophore is the donor and the tetrapyrrole backbone the acceptor in the FRET pair. Thus, fluorescence from the coumarin following excitation of the coumarin is quenched in compounds 1, 2, C3 and C4.

TABLE 6

| Number | Structure |
|---|---|
| 1 | 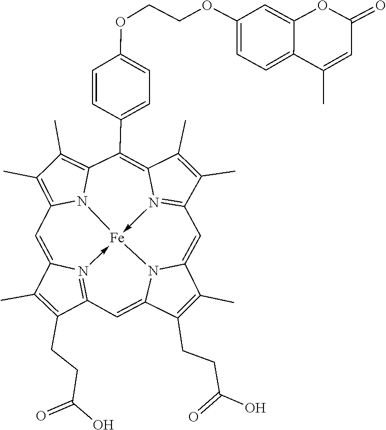 |
| 2 | 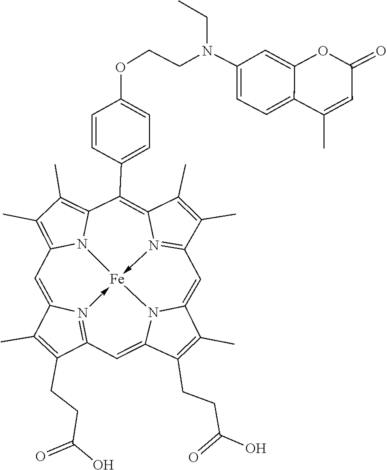 |
| C3 | 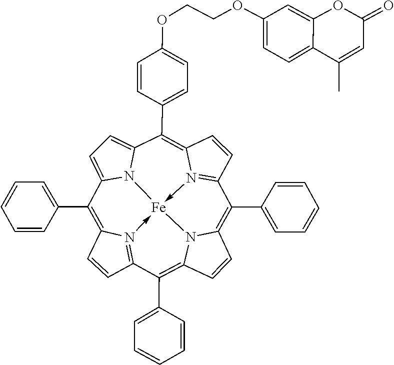 |
| C4 | 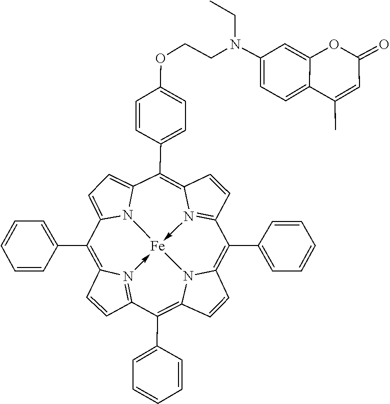 |

1 and 2 include the same porphyrin backbone based on Fe-DMD. C3 and C4 are based on the structure of Fe-HP-TPP, which does not fall within the scope of the present invention.

The synthetic route to 1 is shown in Scheme 1 below. In summary, this synthesis was performed starting with benzyl 3,4-dimethyl-1H-pyrrole-2-carboxylate 1a. Condensation of 1a with 4-hydroxybenzaldehyde in dichloromethane and trifluoracetic acid formed 1b. Subsequent benzyl ester hydrogenation produced 1c and MacDonald condensation of 1c with di-formyl dipyrromethene 1d produced 1e. Alkylation of 1e with 1f was achieved in DMF at room temperature over 5 d with potassium carbonate as the base, forming 1g. Complexation of 1g with FeCl₂·4H₂O in chloroform/methanol (3:1) produced lh and the methyl ester hydrolysis under basic conditions of 1h gave rise to 1 after lyophilisation.

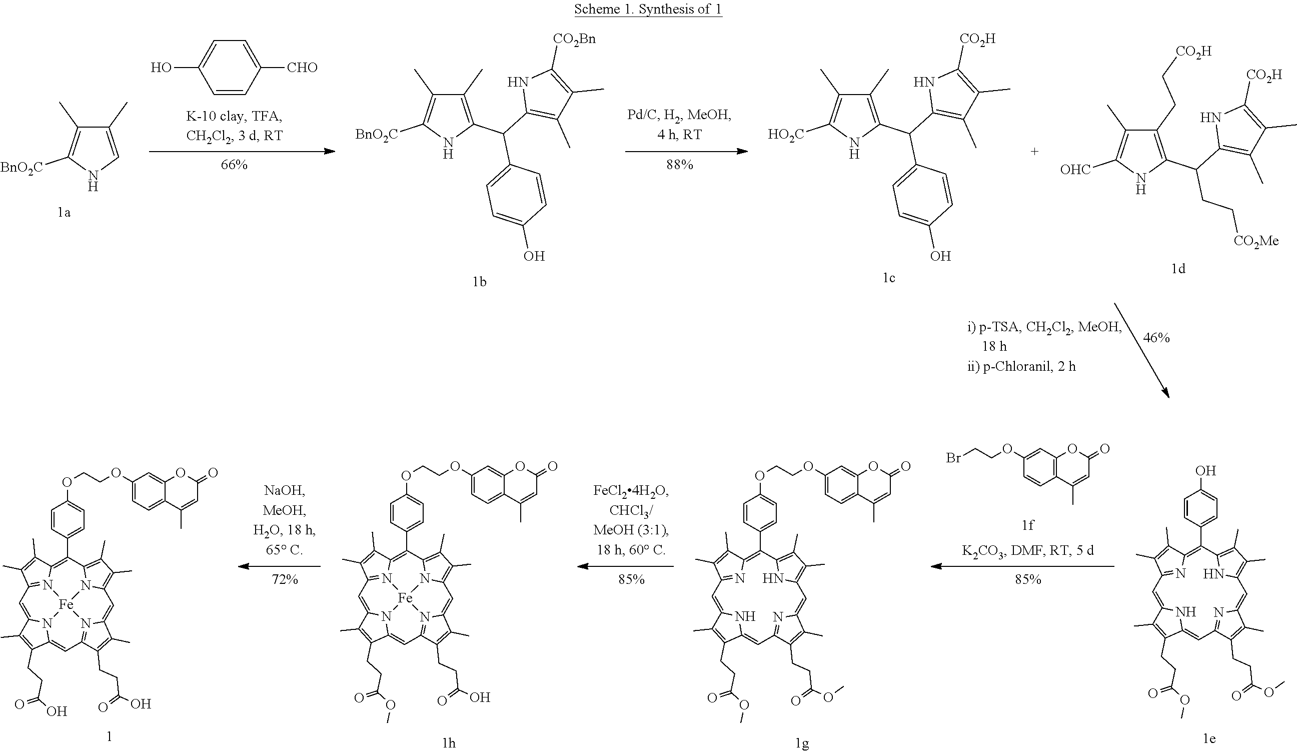

General Procedures: All commercially available reagents were used as received from suppliers without further purification. Solvents used were laboratory grade. Anhydrous solvents were obtained from departmental solvent towers and stored over 3 Å molecular sieves. Moisture-sensitive reactions were carried out by Schlenk-line techniques, under an inert atmosphere of nitrogen. Thin-layer and column chromatography was performed on silica (Merk Art 5554) and visualised under UV radiation. ¹H (400 MHz) and ¹³C {H} (101 MHz) NMR spectra were recorded on a Bruker AV-400 spectrometer, Imperial College London at 298 K. Chemical shifts are reported in parts per million (ppm) and coupling constants in Hertz (Hz). Peak multiplicities are abbreviated as; s=singlet, m=multiple, d=doublet, t=triplet, q=quartet, dd=doublet of doublet and br=broad. Mass spectrometry analysis (ESI and MALDI-MS) was conducted by the Mass Spectrometry Service, Imperial College London, unless stated otherwise.

Compounds 1a, 1d and 1f were prepared using procedures from the literature, as described in Lash, T. D.; Bellettini, J. R.; Bastian, J. A.; Couch, K. B. Synthesis of Pyrroles from Benzyl Isocyanoacetate. Synthesis (Stuttg). 1994, 170-172; Martin, P.; Mueller, M.; Flubacher, D.; Boudier, A.; Blaser, H. U.; Spielvogel, D. Total Synthesis of 20 Hematoporphyrin and Protoporphyrin: A Conceptually New Approach. Org. Process Res. Dev. 2010, 14, 799-804; and Jiang, N.; Huang, Q.; Liu, J.; Liang, N.; Li, Q.; Li, Q.; Xie, S. S. Design, Synthesis and Biological Evaluation of New Coumarin-Dithiocarbamate Hybrids as Multifunctional Agents for the Treatment of Alzheimer's Disease. Eur. J. Med. Chem. 2018, 146, 287-298 respectively.

Dibenzyl-5,5′-(4-hyroxyphenyl)methylene)bis(3,4-dimethyl-1H-pyrrole-2-carboxylate)-1b.

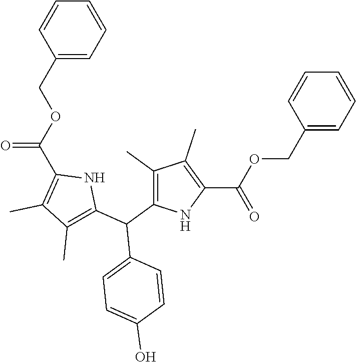 [SMILES: Cc1c(C(=O)OCc2ccccc2)[nH]c(C(c2ccc(O)cc2)c2[nH]c(C(=O)OCc3ccccc3)c(C)c2C)c1C]

K-10 nontmorillonite clay (0.86 g) was added to a solution of 1a (213.7 mg, 0.93 mmol) in anhydrous Ch₃Cl₂ (12 mL). 4-Hydroxybenzaldehyde (60.3 mg, 0.49 mmol) and TFA (1.0 mL, mmol) were added, and the suspension was stirred at room temperature for 2 d under an inert atmosphere of nitrogen. The suspension was diluted with Ch₃Cl₂ (10 mL) and insoluble inorganics were removed by filtration, and washed Ch₃Cl₂ (2×10 mL). Organic extracts were combined and washed with NaHCO₃ (saturated solution, 20 mL) and brine (20 mL) and dried over Na₂SO₄. The solvent was removed under reduced pressure to form a dark red solid. Purification by silica gel column chromatography (gradient 100% hexane to 60% hexane, 40% ethyl acetate) formed the title compound as a pale pink oil (180.9 mg, 66%). ¹H NMR (400 MHz, CDCl₃) 8.40 (2 H, br s, —NH), 7.39-7.24 (10 H, m), 6.91 (2 H, d, ³J_{H-H} 8.4), 6.74 (2 H, d, ³J_{H-H} 8.4), 5.67 (1 H, br s, —OH), 5.41 (1 H, s), 5.26 (4 H, s), 2.25 (6 H, s), 1.77 (6 H, s), ¹³C {¹H} (101 MHz, CDCl₃) 161.7, 155.2, 136.5, 132.6, 130.4, 129.5, 128.4, 128.0, 127.9, 117.9, 117.4, 116.1, 115.9, 65.7, 40.4, 10.8, 8.9; ESI-LRMS [C₃₅H₃₅N₂O₅]⁺, (+) m/z 563.3, ESI-HRMS calculated for [C₃₅H₃₅N₂O₅]⁺, 563.2546 found, 563.2554.
5,5′-(4-Hyroxypheny)methylene)bis(3,4-dimethyl-1H-pyrrole-2-carboxylic acid)-1c.

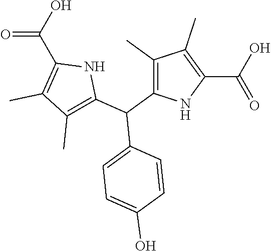 [SMILES: Cc1c(C(=O)O)[nH]c(C(c2ccc(O)cc2)c2[nH]c(C(=O)O)c(C)c2C)c1C]

Palladium on charcoal (10 wt %, 112.4 mg, 0.11 mmol) was added to a solution of compound 1b (492.6 mg, 0.88 mmol) in ethanol (30 mL) and the reaction was stirred at room temperature for 4 h under an atmosphere of hydrogen. After this time, the reaction mixture was filtered through a celite plug, and washed with ethanol (20 mL) and Ch₃Cl₂ (20 mL). The solvent was removed under reduced pressure to form a purple oil/solid (287.5 mg, 88%). ¹H NMR (400 MHz, MeOD) 6.88 (2 H, d, ³J_{H-H} 6.9), 6.74 (2 H, d, ³J_{H-H} 6.9), 5.56 (1 H, s), 2.25 (6 H, s), 1.81 (6 H, s), ¹³C {¹H} (101 MHz, MeOD) 163.1, 156.2, 133.1, 130.4, 129.4, 129.0, 127.6, 117.3, 115.1, 39.6, 9.4, 7.5; ESI-LRMS [C₂₁H₂₁N₂O₅]⁻, (−) m/z 381.1, ESI-HRMS calculated for [C₂₁H₂₁N₂O₅]⁻, 381.1450 found, 381.1448.
Dimethyl-3,3′-(10-(4-hydroxyphenyl)-3,7,8,12,13,17-hexa-methylporphyrin-2,18-diyl)dipropionate-1e.

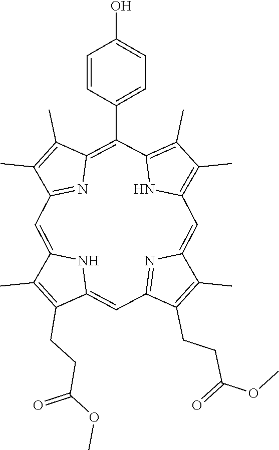

Compound 1c (423.2 mg, 1.11 mmol) was added to a solution of ld (456.4 mg, 1.11 mmol) in anhydrous Ch₃Cl₂ (150 mL). A solution of p-toluene sulfonic acid (758.5 mg, 3.99 mmol) in methanol (20 mL) was added dropwise over 15 min and the solution was stirred at room temperature overnight with protection from light under an inert atmosphere of nitrogen. After 18 h, p-chloranil (545.2 mg, 2.22 mmol) was added and the reaction was stirred for an additional 2 h, before the solvent was removed under reduced pressure to form a dark red residue. Purification silica gel column chromatography (gradient 100% CHCl₃ to 95% CHCl₃/5% MeOH) formed the title compound as a dark red solid (344.8 mg, 46%). ¹H NMR (400 MHz, CDCl₃) 10.1 (2 H, s), 9.96 (1 H, s), 7.06 (2

H, d, ³J_{H-H} 7.9), 6.68 (2 H, ³J_{H-H} 7.9), 4.42 (4 H, t, ³J_{H-H} 7.7), 3.70 (6 H, s), 3.67 (6 H, s), 3.46 (6 H, s), 3.34 (4 H, t, ³J_{H-H} 7.7), 2.10 (6 H, s), ¹³C {¹H} (101 MHz, CDCl₃) 173.8, 158.1, 156.1, 144.9, 144.8, 143.0, 137.8, 137.6, 137.2, 136.9, 133.7, 118.5, 114.2, 96.8, 95.2, 51.8, 37.0, 22.0, 14.9, 12.1, 11.8; ESI-LRMS [C₄₀H₄₃N₄O₅]⁺, (−) m/z 659.3, ESI-HRMS calculated for [C₄₀H₄₃N₄O₅]⁺, 659.3233 found, 659.3245; UV-Vis (CHCl₃, λₘₐₓ/nm): (ε/M⁻¹ cm⁻¹) 405 (100179), 504 (14248), 537 (8943), 571 (7123), 624 (3287).

Dimethyl 3,3′-(3,7,8,12,13,17-hexamethyl-10-(4-(244-methyl-2-oxo-2H-chromen-7-yl)oxy)ethoxy) phenyl)porp-hyrin-12, 18-diyl)dipropionate-1g

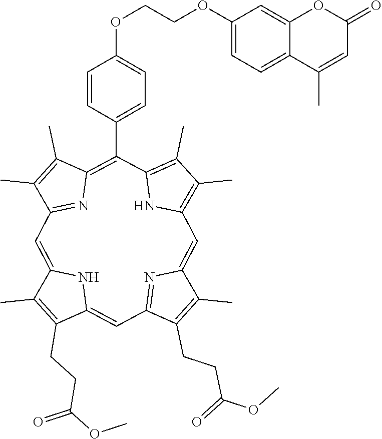 [SMILES: COC(=O)CCC1=C(C)c2cc3[nH]c(c(C)c3C)c(-c3ccc(OCCOc4ccc5c(C)cc(=O)oc5c4)cc3)c3nc(cc4[nH]c(cc1n2)c(CCC(=O)OC)c4C)C(C)=C3C]

Anhydrous K₂CO₃ (56.2 mg, 0.41 mmol) was added to a solution of 1e (26.2 mg, 0.04 mmol) and If (26.3 mg, 0.09 mmol) in anhydrous DMF (2.5 mL). The reaction was stirred at room temperature under an inert atmosphere of nitrogen with protection from light for 4 d. The solvent was removed under reduced pressure to form a dark brown residue. Purification by silica gel column chromatography (gradient 100% Ch₃Cl₂ to 99% CH₂Cl₂/1% MeOH) formed the title compound as a dark red solid (23.5 mg, 68%). ¹H NMR (400 MHz, CDCl₃) (2 H, s), 9.95 (1 H, s), 7.77-7.74 (2 H, m), 7.52-7.44 (1 H, m), 7.10-7.05 (2 H, m), 6.94-6.90 (2 H, m), 6.17 (1 H, br s), 4.44-4.33 (8 H, m), 3.68 (6 H, s), 3.65 (6 H, s), 3.52 (6 H, s), 3.30 (4 H, t, ³J_{H-H} 7.8), 2.41-2.30 (9 H, m); ¹³C {¹H} (101 MHz, CDCl₃) 173.7, 161.6, 161.3, 158.7, 155.2, 152.5, 145.1, 144.9, 143.5, 143.0, 138.1, 137.8, 137.2, 137.0, 135.5, 133.8, 125.6, 118.5, 113.9, 113.6, 112.7, 112.2, 101.6, 96.86, 95.4, 67.1, 66.3, 51.8, 37.0, 21.9, 18.7, 15.2, 12.2, 11.8; ESI-LRMS [C₅₂H₅₃N₄O₈]⁺, (+) m/z 861.4, ESI-HRMS calculated for [C₅₂H₅₃N₄O₈]⁺, 861.3863 found, 861.3887; UV-Vis (CHCl₃, λₘₐₓ/nm): (ε/M⁻¹ cm⁻¹) 291 (14481.4), 321 (23963.1), 405 (113969.6), 503 (10264.3), 538 (5047.8), 572 (4529.2), 625 (1601.4).

Iron(11) dimethyl 3,3′-(3,7,8,12,13,17-hexamethyl-10-(4-(2-((4-methyl-2-oxo-2H-chromen-7-yl)oxy)ethoxy) phenyl)porp-hyrin-2,18-diyl)dipropionate-1h

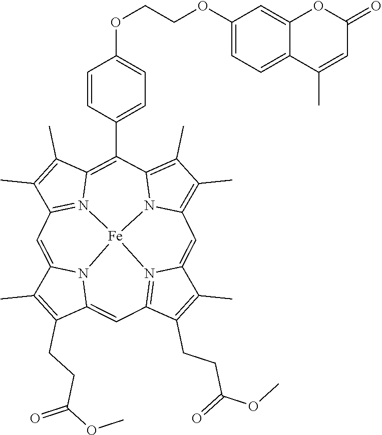

FeCl₂·4H₂O (85.2 mg, 0.43 mmol), sodium hydrogen carbonate (30.2 mg, 0.36 mmol) and sodium ascorbate (21.0 mg, 0.11 mmol) were added to a solution of lg (42.2 mg, 0.05 mmol) in CHCl₃/MeOH (2:1). The solution was heated to 60° C. and stirred under an inert atmosphere of nitrogen with protection from light. After 18 h, the reaction was cooled to room temperature and the solvent was removed under reduced pressure. The crude residue was re-dissolved in Ch₃Cl₂ (20 mL) and washed with H₂O (10 mL) and brine (10 mL). Organic extracts were combined and dried over Na₂SO₄, and the solvent was removed under reduced pressure to form a dark red/brown residue. Purification by silica gel column chromatography (gradient 100% Ch₃Cl₂ to 95% CH₂Cl₂/5% MeOH) formed the title compound as a dark red/brown solid (38.1 mg, 85%). MALDI-MS 914.8; UV-Vis (CHCl₃, λₘₐₓ/nm) : (ε/M⁻¹ cm⁻¹) 290 (28315.0), 321 (34896.4), 384 (58787.9), 506 (8568.1), 535 (7306.8), 638 (2916.6).).

### Compound 1

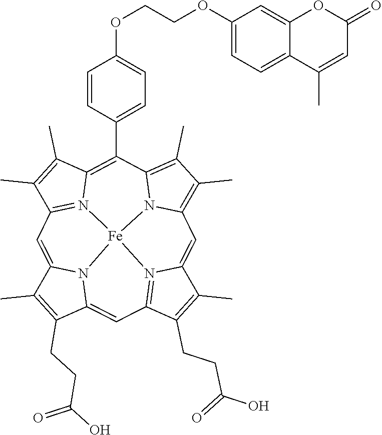

NaOH (4 M, 0.45 mL) was added to a solution of 1 h dimethyl ester in Ch₃Cl₂ (1 mL) and methanol (3 mL). The solution was heated to 40° C. and stirred under an inert atmosphere of nitrogen for 24 h. During the reaction a dark red/brown precipitate appeared indicating the formation of 1. After 24 h the reaction was cooled to room temperature and the solvent was removed under reduced pressure to form a dark brown residue. The residue was re-dissolved in H₂O (5 mL) and acidified to pH 3-4 with HCI (6 M). The precipitate was isolated by centrifuge and washed with H₂O (3×10 mL) and lypholised to form the title compound as a dark purple/brown solid (12.6 mg, 72%). MALDI-MS 886.2; UV-Vis (CHCl₃, λₘₐₓ/nm): ((ε/M⁻¹ cm⁻¹) 320 (19980.8), 385 (59877.1), 523 (4975.3), 548 (4183.6), 640 (2615.6).

The synthesis of 2 was identical to that above for 1 excepting the 7-hydroxymethyl-coumarin derivative If used to alkylate the phenol-substituted porphyrin was replaced by a 7-aminomethyl-coumarin derivative 2f. Compound 2f was reacted with le to form 2g, complexed with Fe²⁺ to form 2 h, and deprotected to form 2.The synthesis for the 7-aminocoumarin derivative (2f) is from: Lin, Q et al., J. Am. Chem. Soc. 2012, 134, 11, 5052-5055. (with the experimental detailed in the supporting information of the above paper).

The synthesis of C3 and C4 were identical to that of 1 and 2 respectively, excepting that the alkylation reaction was performed on C3e in place of le. In the synthesis of C3, C3e was reacted with If to form C3g and complexed with Fe²⁺ to form C3. In the synthesis of C4, C3e was reacted with 2f to form C4g and complexed with Fe²⁺ to form C4.

HO-1 catabolism of 1 is predicted to produce coumarin li as a by-product accompanying biliverdin/bilirubin and F²⁺. This is based on the observation that HO-1 mediated catabolism of phenyl substituted heme produces benzoic acid, Wang, J et al., J. Biol. Chem., 2004, 279, 41, 42593-42604. In order to quantify the degradation product formed, compound 1 i was synthesised in a two-step procedure from ethyl 4-hydroxybenzoate. This is shown in Scheme 2 below.

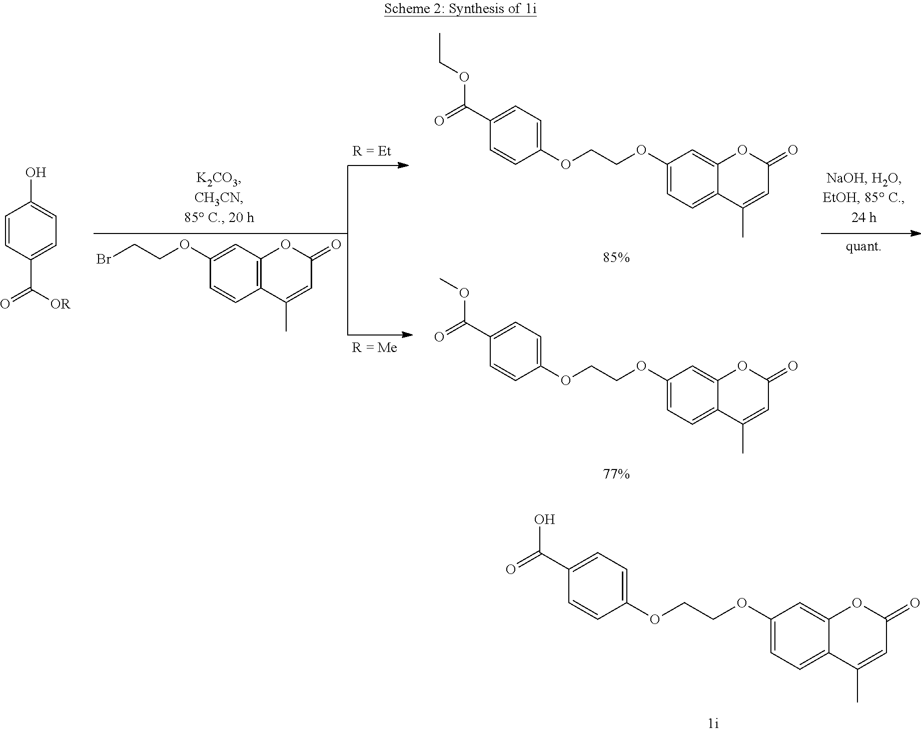

Ethyl 4-(2-((4-methyl-2-oxo-2H-chromen-7-yl)oxy)ethoxy)benzoate

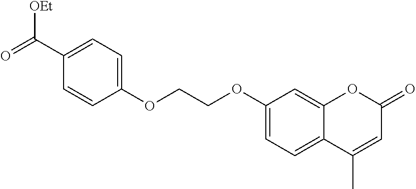 [SMILES: CCOC(=O)c1ccc(OCCOc2ccc3c(C)cc(=O)oc3c2)cc1]

Coumarin 1f (165.2 mg, 0.58 mmol) was added to a stirring solution of ethyl-4-hydroxybenzoate (80.2 mg, 0.48 mmol), potassium carbonate (300.4 mg, 2.17 mmol) and potassium iodide (7.0 mg, 0.04 mmol) in anhydrous acetonitrile (7 mL). The reaction was heated to reflux and stirred for 24 h, before being cooled to room temperature. The suspension was diluted with Ch₃Cl₂ (20 mL) and filtered to remove any inorganic impurities and the solvent was removed under reduced pressure to form an off-white solid.

Purification by column chromatography (gradient 100% hexane to 100% CH 2 01 2) formed the title compound as a white crystalline solid (150.9 mg, 85%). ¹H NMR (400 MHz, CDCl₃) 8.03-7.99 (2 H, m), 7.51 (1 H, d, ³J_{H-H} 8.8), 6.98-6.95 (2 H, m), 6.92 (1 H, dd, ³J_{H-H} 8.8 and ⁴J_{H-H} 2.6), 6.87 (1 H, d, ⁴J_{H-H} 2.6), 6.15 (1 H, d, ⁴J_{H-H} 1.3), 4.40 (4 H, s), 4.34 (2 H, q, ³J_{H-H} 7.1), 2.40 (3 H, d, ⁴J_{H-H} 1.3), 1.38 (3 H, t, ³J_{H-H} 7.1); ¹³C {¹H} (101 MHz, CDCl₃) 166.3, 162.1, 161.5, 161.2, 155.2, 152.5, 131.6, 125.7, 123.6, 114.2, 114.0, 112.7, 112.3, 101.6, 66.9, 66.3, 60.7, 18.7, 14.4; ESI-LRMS [C₂₁H₂₁O₆]⁺, (+) m/z 369.1; ESI-HRMS calculated for [C₂₁H₂₁O₆]⁺, 369.1333 found, 369.1324.

Methyl 4-(2-((4-methyl-2-oxo-2H-chromen-7-yl)oxy)ethoxy)benzoate

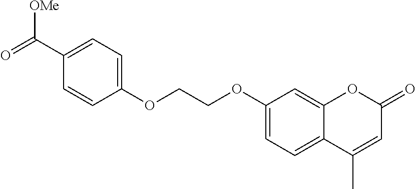 [SMILES: COC(=O)c1ccc(OCCOc2ccc3c(C)cc(=O)oc3c2)cc1]

Coumarin 1f (239.2 mg, 0.84 mmol) was added to a stirring solution of methyl-4-hydroxybenzoate (104.6 mg, 0.69 mmol), potassium carbonate (554.0 mg, 4.0 mmol) and potassium iodide (12.1 mg, 0.07 mmol) in anhydrous acetonitrile (8 mL). The reaction was heated to reflux and stirred for 24 h, before being cooled to room temperature. The suspension was diluted with Ch₃Cl₂ (20 mL) and filtered to remove any inorganic impurities and the solvent was removed under reduced pressure to form an off white solid. Purification by column chromatography (gradient 100% hexane to 100% CH 2 01 2) formed the title compound as a white crystalline solid (186.4 mg, 77%). ¹H NMR (400 MHz, CDCl₃) 8.05-8.01 (2 H, m), 7.52 (1 H, d, ³J_{H-H} 8.7), 7.01-6.98 (2 H, m), 6.92 (1 H, dd, ³J_{H-H} 8.7 and ⁴J_{H-H} 2.4), 6.88 (1 H, d, ⁴J_{H-H} 2.4), 6.16 (1 H, d, ⁴J_{H-H} 1.3), 4.41 (4 H, s), 3.89 (3 H, s), 2.41 (3 H, d, ³J_{H-H} 1.3); ¹³C {¹H} (101 MHz, CDCl₃) 166.9, 162.3, 161.6, 161.3, 155.3, 155.6, 131.8, 125.8, 123,3, 114.3, 114.1, 112.8, 112.4, 101.7, 67.0, 66.4, 52.1, 18.8; ESI-LRMS [C₂₀H₁₉O₆]⁺, (+) m/z 355.1; ESI-HRMS calculated for [C₂₀H₁₉O₆]⁺, 355.1182 found, 355.1177.

4-(2-((4-Methyl-2-oxo-2H-chromen-7-yl)oxy)ethoxy)benzoic acid,

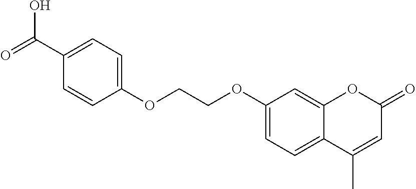 [SMILES: Cc1cc(=O)oc2cc(OCCOc3ccc(C(=O)O)cc3)ccc12]

NaOH (4 M, 0.7 mL) was ded to a solution of coumarin methyl 4-(2-((4-methyl-2-oxo-2H-chromen-7-yl)oxy)ethoxy)benzoate (74.8 mg, 0.20 mmol) in ethanol (5 mL). The solution was heated at 85° C. for h, with the formation of a white precipitate. The precipitate was dissolved in deionised water (5 mL) and acidified to pH 4-5 with 6 M HCI. The white precipitate formed on acidification was isolated by centrifuge, washed with water (3×10 mL) and dried under reduced pressure to form the title compound as a white solid (63.8 mg, 94%). ¹H NMR (400 MHz, (CH₃)₂ SO) 12.60 (1 H, br s), 7.92-7.88 (2 H, m), 7.70 (1 H, d, ³J_{H-H} 8.8), 7.10-7.06 (3 H, m), 7.02 (1 H, dd, ³J_{H-H} 8.8 and ⁴J_{H-H} 2.5), 6.22 (1 H, d, ⁴J_{H-H} 1.3), 4.48-4.42 (4 H, m), 2.40 (3 H, d, ⁴J_{H-H} 1.3); ¹³C {¹H} (101 MHz, (CH₃)₂SO) 167.0, 161.8, 161.3, 160.1, 154.7, 153.4, 131.4, 126.5, 123.3, 114.4, 113.3, 112.5, 111.3, 101.4, 67.0, 66.4, 18.2; ESI-LRMS [C₁₉H₁₇O₆]⁺, (+) m/z 341.1; ESI-HRMS calculated for [C₁₉H₁₇O₆]⁺, 341.1025 found, 341.1024.

### Example 2—Photophysical Studies

UV-Visible absorption spectra were measured using an Agilent Technologies Cary 60 Spectrophotometer operating with VVinUV software. The sample was held in a quartz cuvette with a path length of 1 cm. Absorption spectra were recorded against a baseline of pure solvent in an optically matched cuvette with a scan rate of 600 nm/min and a data interval of 1.0 nm. Emission and excitation spectra were acquired on an Agilent Technologies Carry Eclipse Fluorescence Spectrophotometer, in quartz cuvettes with a path length of 1 cm. Emission and excitation spectra were collected with a scan rate of 120.0 nm/min, a delay interval of 1.0 nm and band-passes of 5 nm.

The photophysical properties of 1i, 1, and C3 and their precursors are shown in Table 7. Measurements were recorded in aqueous solutions of PBS buffer at pH 7.4 in order to mimic physiological conditions. Table 8 shows the photophysical properties of 1i, 1, and C3 and their precursors measured in chloroform at 298 K.

TABLE 7
Photophysical data for compounds 1i, 1, and C3 and their precursors in
PBS buffer pH 7.4. Concentration = 20 μM in PBS buffer (pH =
7.4), λₑₓ = 320 nm, 298 K. Quantum yields (Ø) ± 20% were measured
using tetraphenylporphyrin (H₂TPP) in toluene (Ø_{514 nm} = 0.11) as the standard.

| | PBS buffer pH 7.4 | | | | | |
|---|---|---|---|---|---|---|
| | λ_{abs} [nm] | | | | | |
| | (ε/10⁴ M⁻¹cm⁻¹) | | | Emission | | |
| Ligand | UV | Soret | Q-band | λₘₐₓ [nm] | E [%] | Ø_{514 nm} [%] |
| 1f | 320 (1.5) | — | — | 383 ^{[b]} | — | —^{[d]} |
| 1g | 320 (1.0) | 413 (1.6) | ^{[a]} | 387, ^{[b]} 638, | 95.1 | 0.5 |
| | | | | 675, 708 | | |
| 1h | 320 (2.2) | 403 (2.5) | ^{[a]} | 387 ^{[b], [c]} | 99.2 | —^{[d]} |
| 1 | 321 (2.2) | 401 (3.0) | ^{[a]} | ^{[b], [c]} | 99.6 | —^{[d]} |
| 1i | 320 (1.5) | — | — | 384 | — | —^{[d]} |
| C3g | 294 (0.6) | 430 (2.7) | 527 (0.4), | 383, ^{[b]} 660, | 96.8 | 2.0 |
| | 320 (0.7) | | | 727 | | |
| | | | 565 (0.2), | | | |
| | | | 603 (0.2) | | | |
| | | | 661 (0.1) | | | |
| C3 | 320 (1.2) | 417 (1.5) | 587 (0.8), | 383 ^{[b] [c]} | 99.4 | —^{[d]} |
| | | | 629 (0.7) | | | |

^{[a]} Broad Q-bands,
^{[b]} residual coumarin emission,
^{[c]} no porphyrin emission was observed,
^{[d]}no quantum yield was measured.

TABLE 8
Photophysical data for compounds 1i, 1 and C3 and their precursors
in CHCI₃, λₑₓ = 320 nm, 298 K. Quantum yields (Ø) ± 20%
were measured using tetraphenylporphyrin
(H₂TPP) in toluene (Ø_{514 nm} = 0.11) as the standard.

| | Chloroform | | | | | |
|---|---|---|---|---|---|---|
| | λ_{abs} [nm] | | | | | |
| | (ε/10⁴ M⁻¹cm⁻¹) | | | Emission | | |
| Ligand | UV | Soret | Q-band | λₘₐₓ [nm] | E [%] | Ø_{514 nm} [%] |
| 1f | 291 (0.9) | — | — | 382 | — | — ^{[c]} |
| | 323 (1.4) | | | | | |
| 1g | 291 (1.4) | 405 (11.4) | 503 (1.0), | 385^{[a]}, 629, | 99.7 | 5.2 |
| | 320 (2.4) | | 538 (0.5), | 695 | | |
| | | | 572 (0.4), | | | |
| | | | 625 (0.2) | | | |
| 1h | 291 (2.8) | 384 (5.9) | 506 (0.9), | 385^{[a], [b]} | 98.7 | — ^{[c]} |
| | 321 (3.5) | | 535 (0.7), | | | |
| | | | 638 (0.3) | | | |
| 1 | 320 (2.0) | 385 (6.0) | 523 (0.5), | 385 ^{[a], [b]} | 99.2 | — ^{[c]} |
| | | | 548 (0.4), | | | |
| | | | 640 (0.3) | | | |
| 1i | 291 (0.9) | — | — | 381 | — | — ^{[c]} |
| | 321 (1.7) | | | | | |
| C3g | 291 (0.9) | 419 (17.6) | 516 (0.7), | 379^{[a]}, 654, | 96.6 | 17.4 |
| | 320 (1.1) | | 552 (0.3), | 719 | | |
| | | | 592 (0.2), | | | |
| | | | 649 (0.3) | | | |
| C3 | 320 (4.1) | 409 (10.0) | 572 (0.8), | 385^{[a], [b]} | 99.8 | — ^{[c]} |
| | | | 618 (0.4) | | | |

^{[a]}Residual coumarin emission,
^{[b]} no porphyrin emission was observed,
^{[c]} no quantum yield was measured.

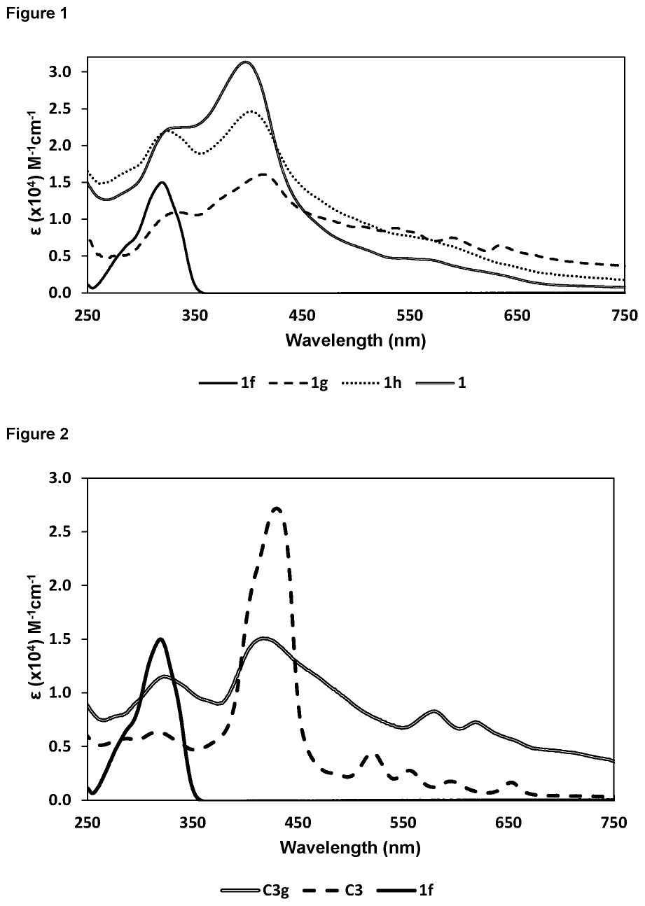

The absorbance spectra of 1, 1f, 1g, 1 h, 1, C3 and C3g in PBS buffer are shown in FIGS. 1 and 2.

In PBS buffer 1 displayed a broad Soret band at 401 nm and a broad Q-band stretching from 541 to 680 nm assigned to S₀→S₂ and S₀→S₁ transitions respectively. The absorbance at 320 nm was assigned to the coumarin moiety containing localized π-π* character. Biologically relevant analogue 1 displayed a significantly broader absorbance spectrum in aqueous media compared to that in chloroform. Such a phenomenon is typical of PPIX analogues, due to the increase in π-stacking, and is strongly dependent on pH and ionic strength. In contrast to 1, compound 3, displayed a sharper absorbance spectra in PBS buffer, and a red-shifted Soret band at 417 nm.

Complexation of Fe²⁺ was confirmed from the change in the number of Q-bands in the UV-Vis spectrum in chloroform — from four to two. A blue-shift in the Soret band was also observed in both aqueous and organic media. For example, 1 h displayed a 10 nm hypsochromic shift after Fe²⁺ complexation.

In PBS, a 52 nm red-shift was observed in the absorbance maximum of the coumarin donor when 7-aminimethyl-coumarin was used (2 and C4) compared to the 7-hydroxymethyl-analogue (1 and C3). Similarly to 1, analogues of compound 2 displayed significantly broader absorbance spectra in aqueous media, typical of PPIX analogues due to the increase in π-stacking as a function of pH and ionic strength.

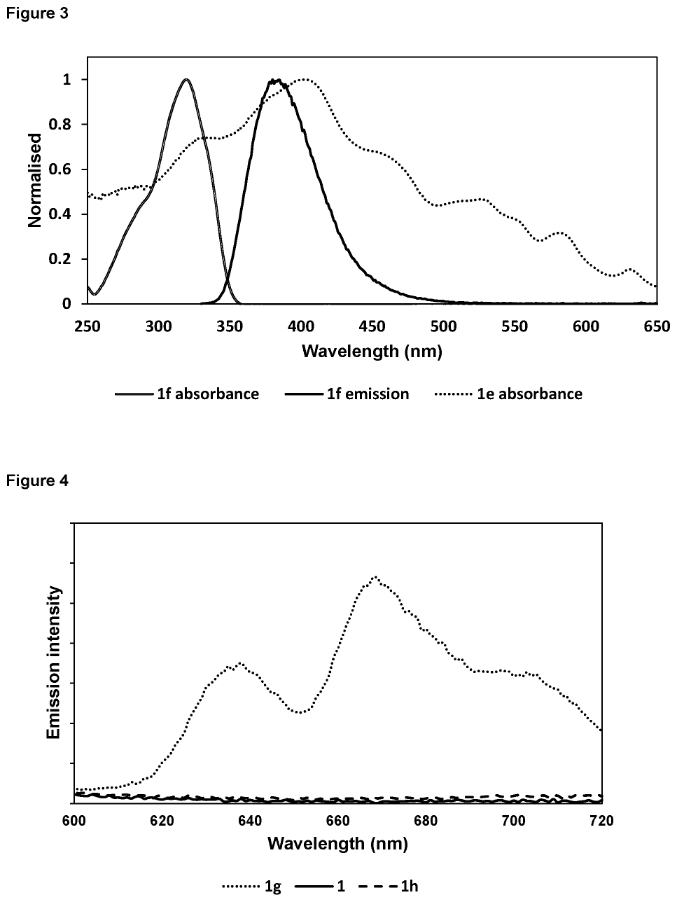

The absorbance spectra of the unmodified porphyrin 1e is shown in FIG. 3 along with the absorbance and emission spectra of 1f. This demonstrations absorption across a broad range of wavelengths from about 300 nm to 500 nm, with emission at red wavelengths.

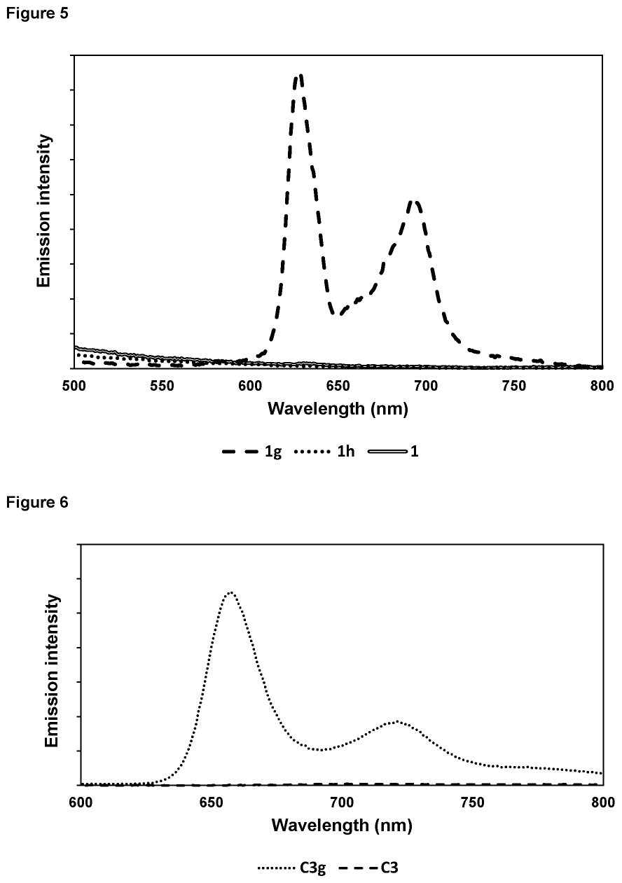

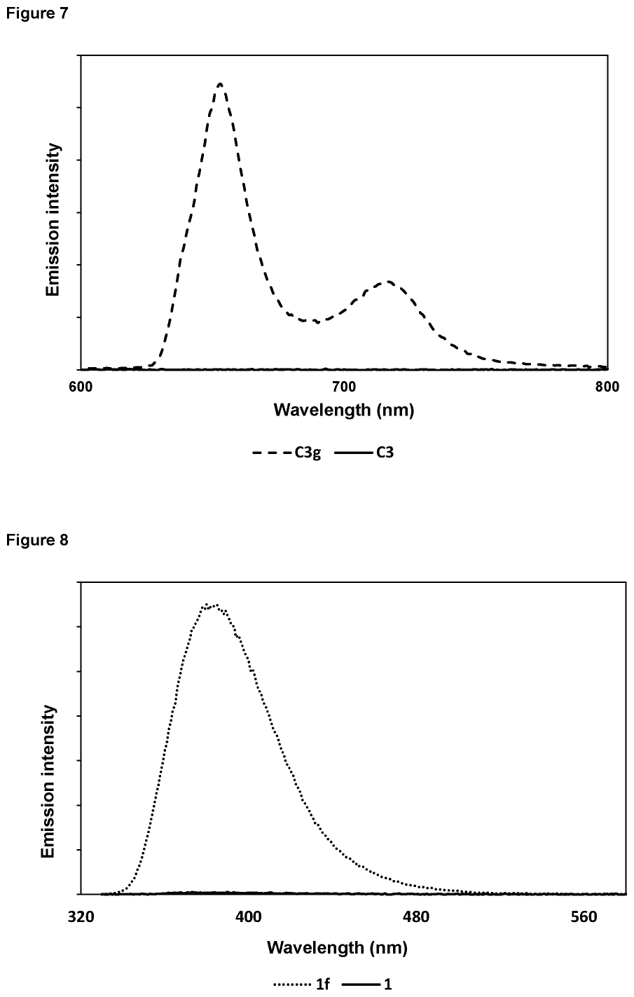

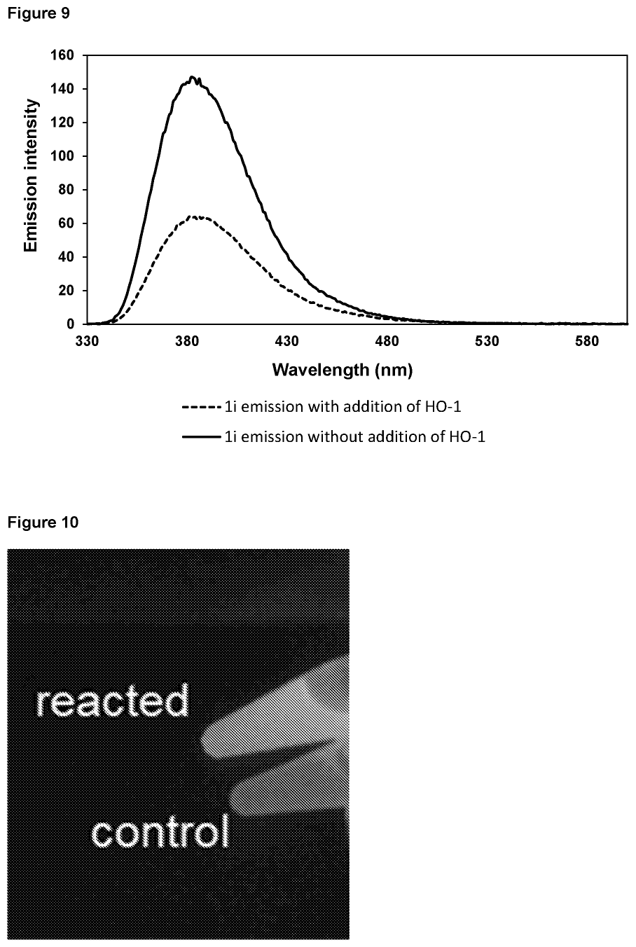

The emission spectra of 1g, 1 h and 1 at Aex=320 nm in PBS buffer pH=7.4 and chloroform are shown in FIGS. 4 and 5 respectively. The emission spectra of C3g and C3 at λₑₓ=320 nm in in PBS buffer pH=7.4 and chloroform are shown in FIGS. 6 and 7 respectively.

In the examples highlighted above, the sample concentration was 20 pM in PBS buffer and 10 pM in chloroform. All measurements were performed at 298 K.

In both PBS and chloroform, all coumarin-porphyrin diads were found to have an excellent FRET efficiency of >95%.

Following excitation at the λₘₐₓ of the coumarin moiety (320 nm), characteristic porphyrin emission spectra was observed in free base analogues 1g and C3g. However, a different emission profile was observed in each case. In PBS buffer, 1g displayed two main peaks at 638 nm and 675 nm with a broad shoulder at 708 nm (FIG. 4 and Tables 7 and 8).

Conversely, C3g displayed a spectra with two distinct emission peaks at 660 nm and 727 nm. This difference in spectral shape is likely to be due to z.-stacking in aqueous media, and this behaviour is not observed in chloroform where both spectra have the same emission profile. In both PBS buffer and chloroform, C3g displays a red-shifted emission vs. 1g due to the increased conjugation provided by the three additional meso-phenyl substituents.

Following Fe²⁺ complexation of the free-base analogues, porphyrin emission was completely quenched in PBS buffer and chloroform (see 1h, 1 and C3 which show emission at the baseline in FIGS. 4 to 7). Such behavior is not surprising, as Fe²⁺ is well known to quench fluorescence through electron and/or energy transfer processes. Complexation of Fe²⁺ also significantly quenches residual coumarin emission. Therefore, prior to porphyrin catabolism by HO-1, no porphyrin fluorescence and only weak coumarin fluorescence was observed.

The emission spectra of 1 and 1f was also determined following excitation at the λₘₐₓ of the coumarin moiety (320 nm), demonstrating emission centered at 384 nm in PBS buffer (FIG. 8). The emission spectra of 1 can be seen at the baseline in FIG. 8.

### Comparative Example3—HO-1 Activity on the Compounds

Fluorescence from the coumarin is quenched in 1, 2, C3 or C4, but will be observed upon break apart of the compound at the 5 position as the coumarin is separated from the tetrapyrrole backbone, preventing FRET between these moieties.

The effect on fluorescence of the presence of HO-1 was determined by adding an E. coli lysate overexpressing human HO-1 to a solution comprising 1 and C3 either with or without 16 h incubation with NADPH (1 mM). Under such conditions, biliverdin is not further converted to bilirubin due to the lack of biliverdin reductase (BVR), therefore, removing the potential interference from the substrate tolerance of BVR.

Compounds 1 and C3 were incubated at 37° C. (310 K) for 16 h with 1 mM NADPH. In order to quantify HO-1 activity, a control experiment was carried out where 1 and C3 were incubated in E. coli lysate without the addition of NADPH and without incubation, a requirement for HO-1 heme catabolism to take place.

It was found that HO-1 significantly quenched the fluorescence of coumarin fluorophore 1i (the expected break apart product following porphyrin cleavage at the 5 position), with control experiments on 1i demonstrating a 2.3-fold decrease in the emission intensity at 384 nm following addition of HO-1 to compound 1i, at λₑₓ=320 nm in E coli lysates as shown in FIG. 9.

Thus, in order to eliminate the quenching effect of HO-1 in the E. coli lysate system, following incubation acidification (5% v/v H₂SO₄ in methanol) of the break apart products was performed, followed by extraction into chloroform. HO-1 was found to remain in the aqueous phase and was thus effectively removed from the reaction products.

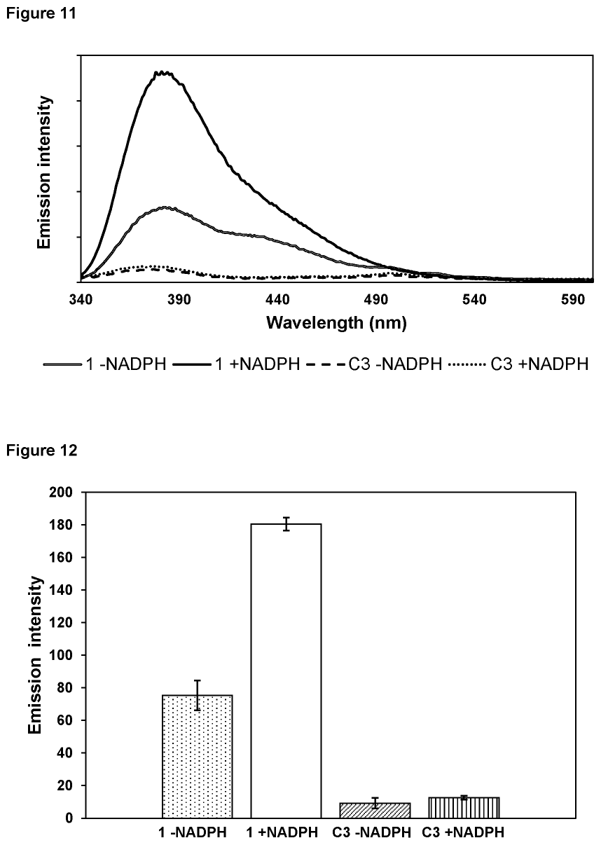

Using this method, a 2.5-fold increase in fluorescence at 384 nm was observed following the incubation of probe 1 with NADPH vs. the control (FIGS. 11 and 12) . Measurements were performed in PBS buffer at pH 7.4. Coumarin emission was centred at 384 nm, a wavelength maximum characteristic to that of coumarin 1i at λₑₓ=320 nm in PBS buffer as shown in FIG. 9, indicating the formation of this molecule.

The significant ‘turn-on’ increase in the fluorescence intensity at the emission wavelength of the coumarin donor fluorophore confirms cleavage at the 5 position of the porphyrin and the elimination of the coumarin-porphyrin FRET system.

In contrast to 1, very little fluorescence enhancement was observed for C3 when compared to the control (<1.4-fold), at λₑₓ=320 nm in PBS buffer pH=7.4, as shown in FIGS. 11 and 12. This demonstrates that the enzyme does not metabolise these probes and confirming that an analogue of Fe-PPIX is an essential requirement in order to maintain activity for HO-1. This supports the conclusion that large substituents at the 10, 15 and may prevent binding of the enzyme and/or that the propionic acid residues at R₄ and R₂ₐ aid with binding of the compound to the active site of HO-1.

Interestingly, the coumarin emission was significantly lower in C3 compared to 1 in the lysate extracts. This behavior was also observed in PBS buffer, where a 3-fold decrease in the coumarin emission was reported for C3 when compared to 1. Due to this enhanced coumarin quenching observed in C3, any HO-1 activity would be expected to cause a greater fluorescence enhancement than 1.

The intensity of emission from the lysate with −NADPH and +NADPH were also determined in the presence of HO-1. It was found that fluorescence from NADPH was low at wavelengths below 400 nm (at which emission from coumarin is observed), thus the observed increase in emission intensity cannot be attributed to the presence of NADPH.

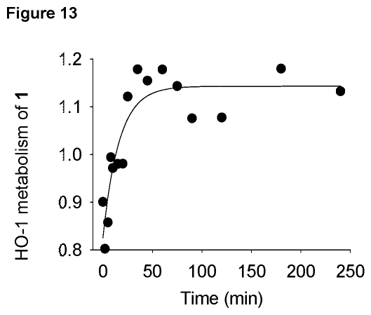

The metabolism of the probes was followed by the appearance of fluorescence from the coumarin fluorophore. A graph for the metabolism of 1 is shown in FIG. 13.

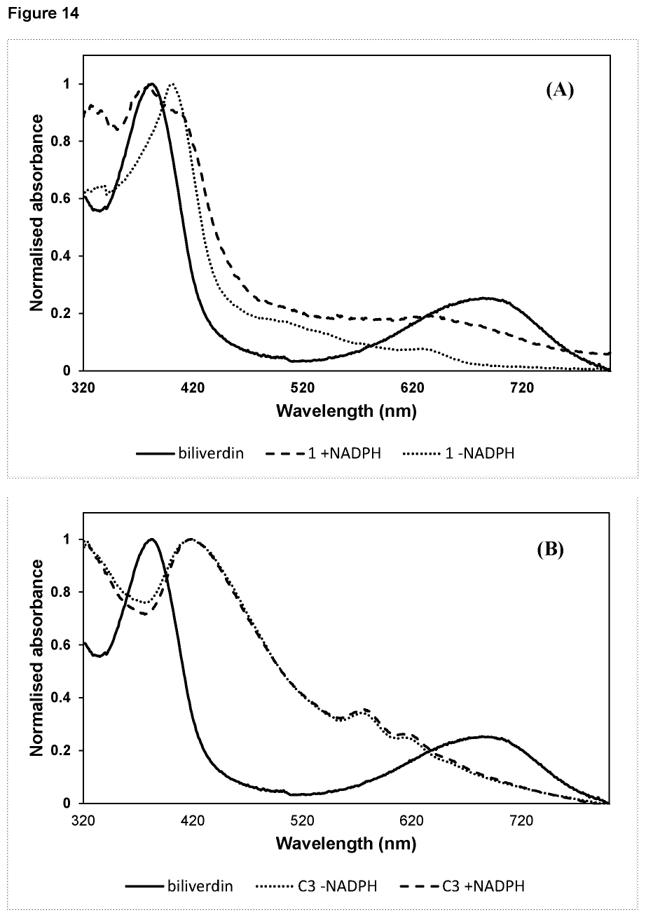

Changes in absorption spectroscopy of 1 following incubation with NADPH in the E. coli lysates overexpressing HO-1 were also monitored, and the resulting spectrum compared to that of the compound without addition of NADPH and biliverdin (the product from metabolism of heme). FIG. 14 (A) shows the absorbance shift to biliverdin (absorbance at about 600 nm to 750 nm) and a 17 nm shift of the Soret band was observed following incubation in the E. coli lysates containing NADPH, indicating successful break apart and release of coumarin. No such shift to biliverdin is observed in FIG. 14 (B) for C3, indicating the poorer ability of HO-1 to metabolise C3.

The effects of HO-1 activity on 1 in the presence and absence of NADPH were also measured using liquid chromatography-mass spectrometry (LCMS), as shown in Figure (A). Acidified samples of E.coli extracts were dissolved in 25% DMSO in acetonitrile to ensure complete solubility and ran from a gradient of 20% -100% -20% acetonitrile in water. The methyl ester of 1i was ran under the same conditions and used as a control. Retention time (R_{T}) is quoted in minutes (min). The methyl ester was hydrolysed to the free acid during the LC-MS analysis. The metabolism of 1 in the presence of HO-1 and NADPH was measured by detection of the coumarin break-down product 1i, which give a characteristic peak of 341 Daltons at a retention time of 12.2 minutes.

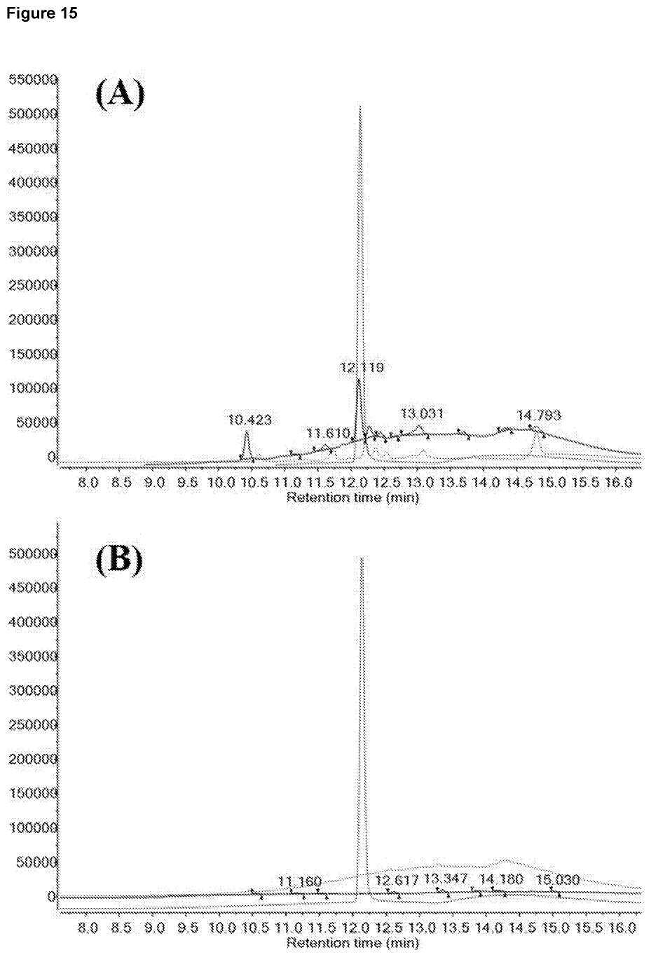

LC-MS experiments were conducted on a solution of the methyl ester of 1i and solutions of 1 in E. coli lysates overexpressing HO-1 either in the presence of NADPH or without as negative control (FIG. 15A). Samples from the E.coli lysates were lyophilised on an Alpha 1-2LD plus −55° C. freeze dryer overnight and subsequently acidified in H₂SO₄/MeOH (5% v/v, 400 μL). The methyl ester of 1i was hydrolysed to the carboxylic acid 1i during acidification.

Each sample was subsequently stirred with a VWR International mini vortex mixer for 30 seconds and left to stand at 298 K for 24 h. After this time, samples were diluted with CHCl₃ (800 μL) and the organic phase was washed with deionised water (3×400 μL). The organic and aqueous layers were separated and the organic solvent was evaporated under atmospheric pressure overnight at 298 K. For fluorescence measurements, samples were dissolved in 20 μL DMSO and diluted with 780 μL PBS buffer (pH=7.4) to a total volume of 800 μL (DMSO content 2.5% v/v). Reaction of 1 with HO-1 in the presence of NADPH resulted in a large increase of the characteristic coumarin 1i peak, demonstrating break apart of the compound. A smaller increase in the characteristic coumarin peak was also observed for the control reaction in the absence of NADPH. No such peak was observed with C3 in the presence of NADPH (FIG. 15 B).

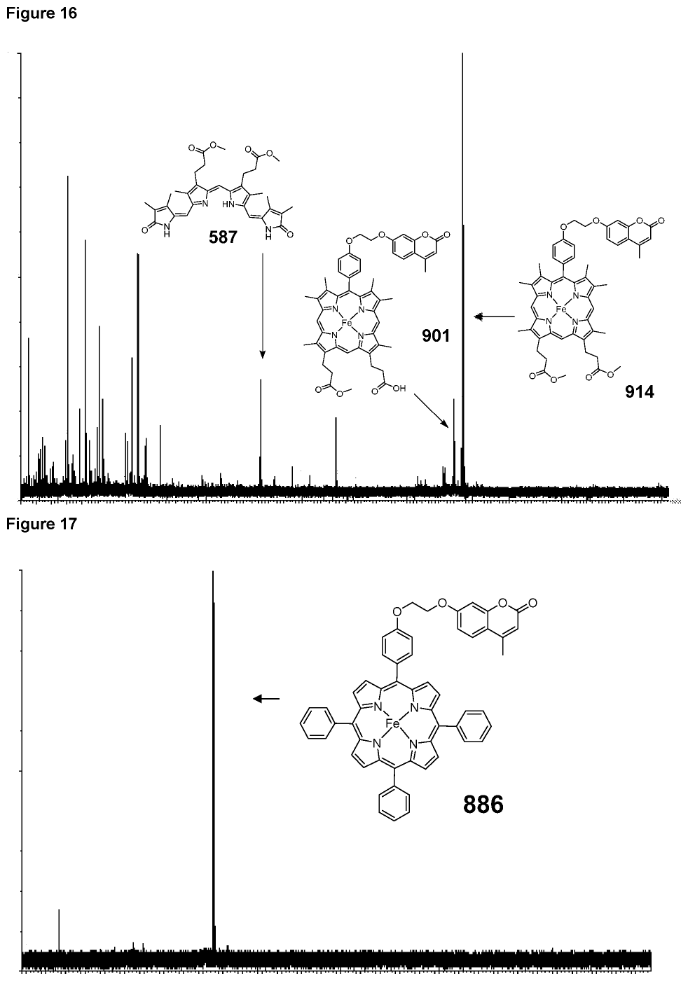

The effects of HO-1 activity on 1 and C3 were also measured using Matrix-assisted laser desorption/ionization mass spectrometry (MALDI MS) Samples from the E.coli lysates were lyophilised on an Alpha 1-2LD plus −55° C. freeze dryer overnight and subsequently acidified in H₂SO₄/MeOH (5% v/v, 400 μL). The methyl ester of 1i was hydrolysed to the carboxylic acid 1i during acidification. Each sample was subsequently stirred with a VWR International mini vortex mixer for 30 seconds and left to stand at 298 K for 24 h. After this time, samples were diluted with CHCI3 (800 pL) and the organic phase was washed with deionised water (3×400 μL).Mass spec samples were submitted as solid samples for MALDI-MS and run by the Mass Spectrometry service at Imperial College London.. MALDI MS experiments were conducted on solutions of either 1 or C3 in the presence of HO-1 and both with and without NADPH. These are shown in FIGS. 16 and 17

In the presence of NADPH and HO-1, the break apart of 1 was evidenced by formation of a biliverdin analogue having a m/z of 587.6. Only one biliverdin analogue was detected which suggests that the cleavage is regiospecific at the 5 position. Methyl and dimethyl analogues of 1 having molecular weights of 900.3 and 914.3 respectively were also detected, suggesting that not all porphyrin was de-cyclised. This is shown in FIG. 16.

Only porphyrin (i.e. no biliverdin analogue) was detected after reaction of 1 and HO-1 in the absence of NADPH.

The reaction of C3 with HO-1 in both the presence and absence of NADPH were measured using MALDI MS. In both experiments, only compound C3 was detected, having an m/z of 886.6, and no porphyrin breakdown products were detected suggesting that very little or no break apart of C3 had taken place. This is shown in FIG. 17 This result demonstrates that C3 is not metabolised by HO-1.

### Example 4—Toxicity

The cell cultures of Example 4 prior to the introduction of HO-1 were compared to control cultures. No toxicity was identified for the compounds of the present invention and appropriate fluorescence emission was identified.

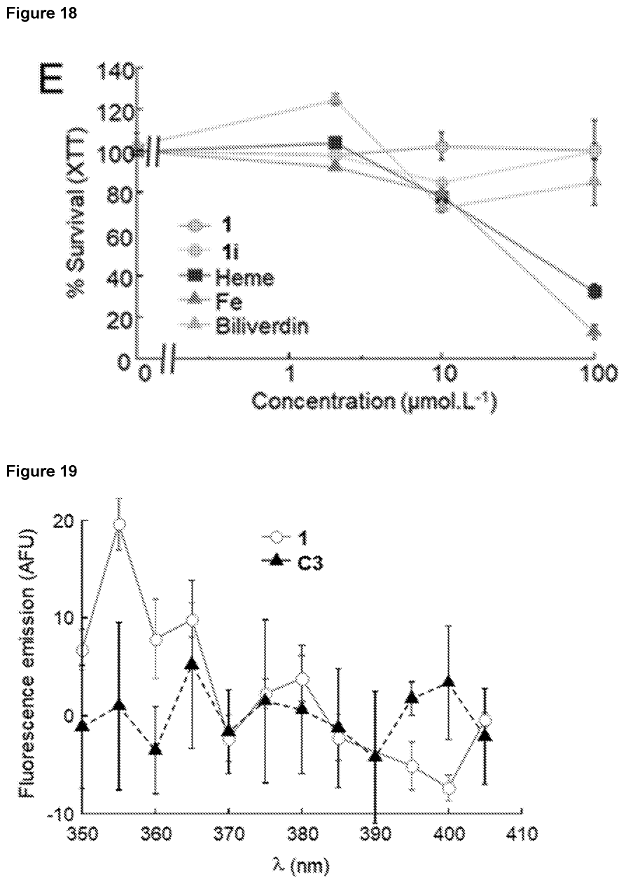

RAW cells were also cultured in the presence of 1, coumarin 1i, heme, iron and biliverdin at a range of concentrations. The compounds according to the present invention were found to be less toxic than endogenous equivalents, as demonstrated by the % survival. These results are shown in FIG. 18.

### Example 5—Heme Oxygenase 1 Detection in Cells

Human monocyte-derived macrophages were purified from blood and cultured in 96-well transparent-bottom black-sided plates for 24 h, then stimulated with 100 nM Trichostatin-A for 6 h. Trichostatin-A is a drug that induces HO-1 but is not optically active. 10 μM compound 1 or C3 was added to the human monocyte derived macrophages for 18 h and the supernatant was changed to remove non-cell-incorporated probe.

After 24 h the fluorescence emission was measured at excitation 323±7nm, these results are shown in FIG. 19. As can be seen, control compound C3 produced no significant change in fluorescence emission from the baseline. However, compound 1 results in a peak of fluorescence emission at 355 nm, corresponding to the coumarin 1i peak demonstrating the break apart of compound 1. Comparisons with the coumarin 1i at the same concentration indicate that approximately 10% of the probe (1 μM) has been converted to the coumarin 1i. The fluorescence datapoints in FIG. 19 are the mean ±SE of n=4 technical replicates, representative of human monocyte-derived macrophages from n=8 subjects.

This experiment demonstrates that compounds of the present invention are able to detect HO-1 in cells.

1. A compound represented by Formula I or a pharmaceutically acceptable salt thereof: 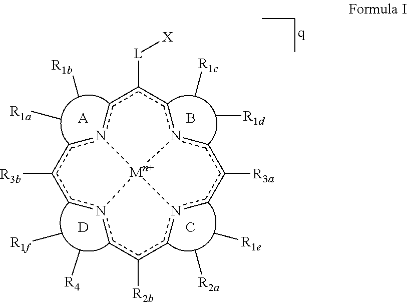
wherein the compound is selected from porphyrins, chlorins, bacteriochlorins or isobacteriochlorins, and wherein: [symbol]
represents a double or single bond; [symbol]
represents a dative or covalent bond between nitrogen and Mⁿ⁺,
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₑ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH2, —OH, —SH, —NH₂, —COORS, —CSOR₅, —COSR₅, —CSSR₅, —CON(R₅)₂, —CONHR₅, —OR ₅, —SRS, —N(R₅)₂, —NHR₅, —CH(OR₅)₂, —CH(OR₅)(SR₅), —CH(SR₅)₂, 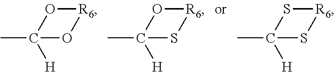
wherein R₅ is monovalent C₁ to C₅-hydrocarbyl or X2, and R₆ is C₁ to C₅alkylene; or R₂ₐ is taken together with R₁ₑ to form a cyclocarbyl;
R₁ₐ and R_{3b} are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂ , —NHR₇—CH(OR₇)₂, —CH(OR₇)(SR₇), —CH(SR₇)₂, 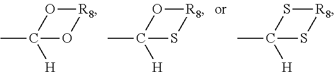
wherein R₇ is monovalent C₁ to C₅-hydrocarbyl or X3, and R₈ is C₁ to C₅-alkylene,
L is a linker;
X, X2 and X3 are fluorophores, which may be the same or different;
Mⁿ⁺ is a cation having a positive charge of integer n, wherein n is from 1 to 3;
q is the overall charge of the compound; and
A, B, C and D each independently represent a five-membered nitrogen-containing heterocycle selected from pyrrole, pyrroline or pyrrolidine rings.

2. The compound according to claim 1, wherein the compound is selected from porphyrins represented by Formula Ha or Ilb or pharmaceutically acceptable salts thereof: 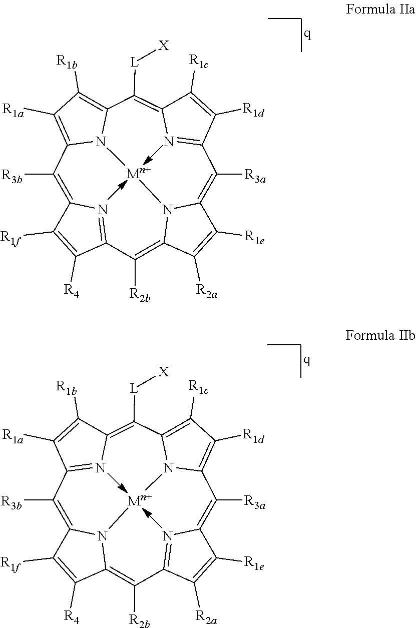
wherein:
→ represents a dative bond between nitrogen and Mⁿ⁺,
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORS, —CSOR5, —COSR5, —CSSR₅, —CON(R₅)₂, —CONHR₅, —OR5, —SRS, —N(Rs)₂, —NHR₅, —CH(OR₅)₂, —CH(OR₅)(SR₅), —CH(SR₅)₂, 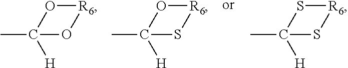
wherein R₅ is monovalent C₁ to C₅-hydrocarbyl or X2, and R₆ is C₁ to C₅ alkylene; or R₂ₐ is taken together with R₁ₑ to form a cyclocarbyl;
R₁ₐ and Rab are independently selected from H, —CH₃, —CH₂CH3, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂, —NHR₇ —CH(OR₇)₂, —CH(OR₇)(SR₇), —CH(SR₇)₂, 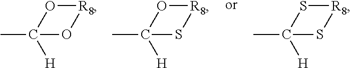
wherein R₇ is monovalent C₁ to C₅-hydrocarbyl or X3, and R₈ is C₁ to C₅-alkylene;
L is a linker;
X, X2 and X3 are fluorophores which may be the same or different;
Mⁿ⁺ is a cation having a positive charge of integer n, wherein n is from 1 to 3; and
q is the overall charge of the porphyrin.

3. The compound according to claim 1, wherein the compound is selected from chlorins represented by any one of Formulas Ma to Ind or pharmaceutically acceptable salts thereof: 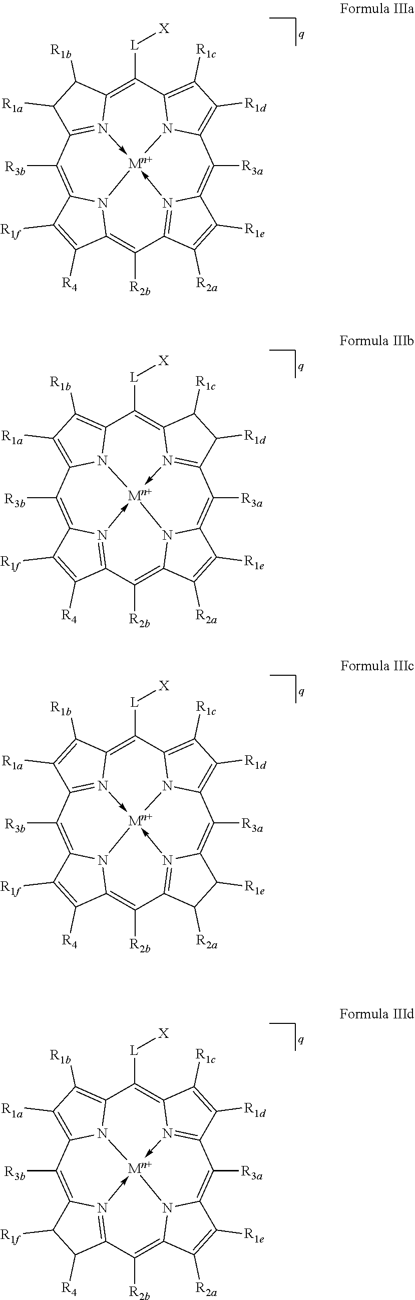
wherein:
→ represents a dative bond between nitrogen and Mⁿ⁺;
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORS, —CSOR₅, —COSR₅, —CSSR₅, —CON(R₅)₂, —CONHR₅, —OR₅, —SR₅, —N(R₅)₂, —NHR₅, —CH(OR₅)₂, —CH(OR₅)(SR₅), —CH(SR₅)₂, 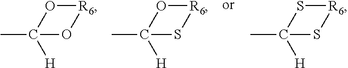
wherein R₅ is monovalent C₁ to C₅-hydrocarbyl or X2, and R₆ is C₁ to C₅ alkylene; or R₂ₐ is taken together with R₁ₑ to form a cyclocarbyl;
R₁ₐ and Rab are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, −SR₇, —N(R₇)₂, —NHR₇ —CH(OR₇)₂, —CH(OR₇)(SR₇), —CH(SR₇)₂, 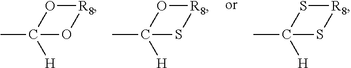
wherein R₇ is monovalent C₁ to C₅-hydrocarbyl or X3, and R₈ is C₁ to C₅-alkylene;
L is a linker;
X, X2 and X3 are fluorophores which may be the same or different;
Mⁿ⁺ is a cation having a positive charge of integer n, wherein n is from 1 to 3; and
q is the overall charge of the chlorin.

4. The compound according to claim 1, wherein the compound is selected from bacteriochlorins represented by Formula IVa or IVb or pharmaceutically acceptable salts thereof: 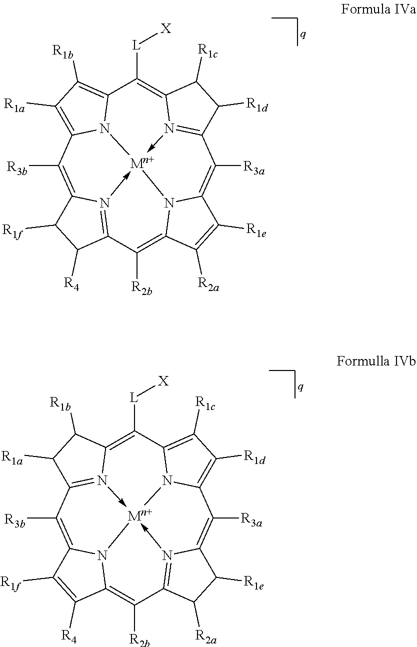
wherein:
→ represents a dative bond between nitrogen and Mⁿ⁺;
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORS, —CSOR₅, —COSR₅, —CSSR₅, —CON(R₅)₂, —CONHR₅, —OR₅, —SR₅, —N(R₅)₂, —NHR₅, —CH(OR₅)₂, —CH(OR₅)(SR₅), —CH(SR₅)₂, 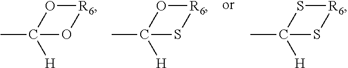
wherein R₅ is monovalent C₁ to C₅-hydrocarbyl or X2, and R₆ is C₁ to C₅ alkylene; or R₂ₐ is taken together with R₁ₑ to form a cyclocarbyl;
R₁ₐ and Rab are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂, —NHR₇ —CH(OR₇)₂, —CH(OR₇)(SR₇), —CH(SR₇)₂, 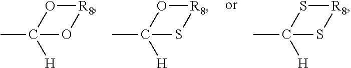
wherein R₇ is monovalent C₁ to C₅-hydrocarbyl or X3, and R₈ is C₁ to C₅-alkylene;
L is a linker;
X, X2 and X3 are fluorophores which may be the same or different;
Mⁿ⁺ is a cation having a positive charge of integer n, wherein n is from 1 to 3; and
q is the overall charge of the bacteriochlorin.

5. The compound according to claim 1, wherein the compound is selected from isobacteriochlorins represented by any one of Formulas Va to Vh or pharmaceutically acceptable salts thereof: 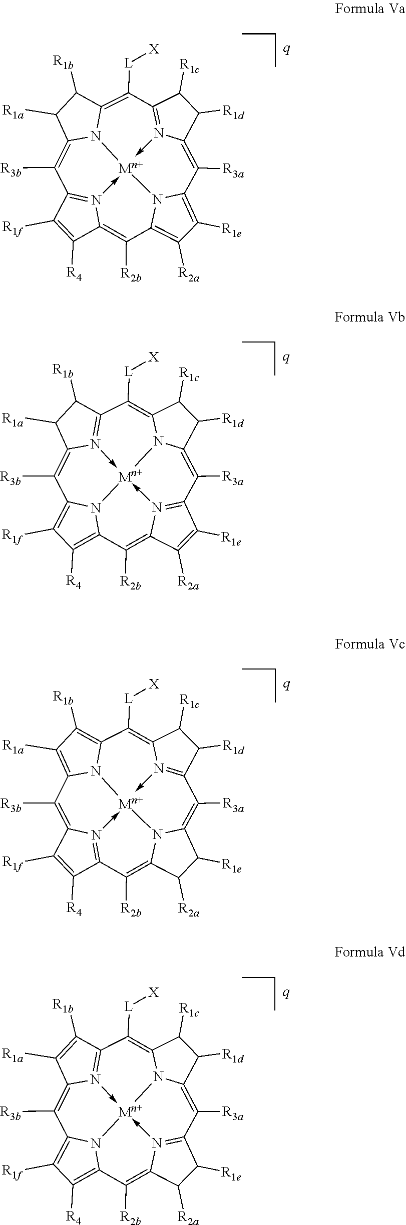 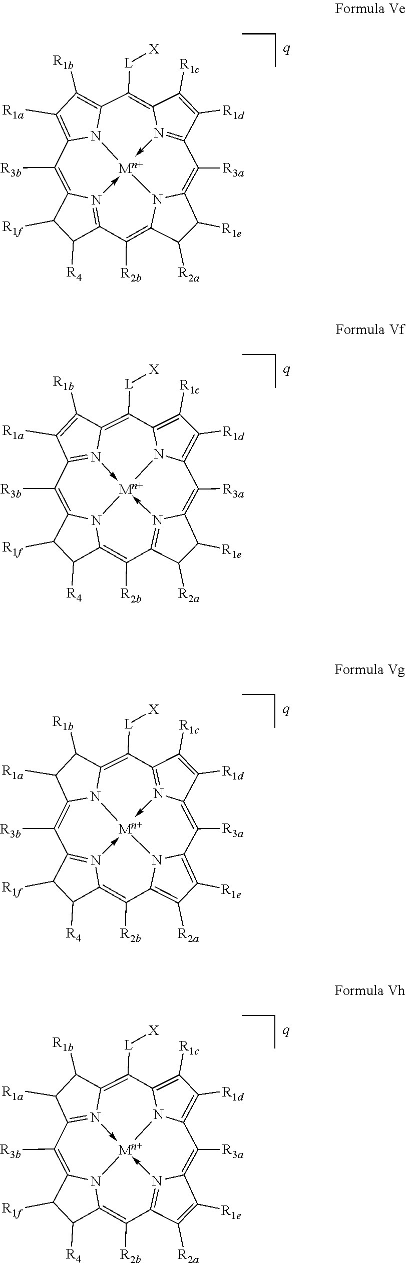
wherein:
→ represents a dative bond between nitrogen and Mⁿ⁺;
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COORs, —CSOR₅, —COSR₅, —CSSR₅, —CON(R₅)₂, —CONHR₅, —OR₅, —SR₅, —N(R₅)₂, —NHR₅, —CH(OR₅)₂, —CH(OR₅)(SR₅), —CH(SR₅)₂, 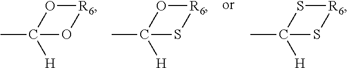
wherein R₅ is monovalent C₁ to C₅-hydrocarbyl or X2, and R₆ is C₁ to C₅ alkylene; or R₂ₐ is taken together with R₁ₑ to form a cyclocarbyl;
R₁ₐ and Rab are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂, —NHR₇ —CH(OR₇)₂, —CH(OR₇)(SR₇), —CH(SR₇)₂, 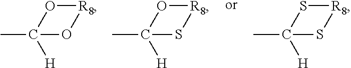
wherein R₇ is monovalent C₁ to C₅-hydrocarbyl or X3, and R₈ is C₁ to C₅-alkylene;
L is a linker;
X, X2 and X3 are fluorophores which may be the same or different;
Mⁿ⁺ is a cation having a positive charge of integer n, wherein n is from 1 to 3; and
q is the overall charge of the isobacteriochlorin.

6. The compound according to claim 1, wherein R₁ₐ to R_{1f} are independently selected from H or monovalent C₁ to C₆-hydrocarbyl, preferably H, C₁ to C₃-alkyl or C₁ to C₃-alkenyl, more preferably H, —CH₃, CH₂CH₃ or —CH═CH2, and most preferably —CH₃.

7. The compound according to claim 1, wherein R₂ₐ and R_{2b} are independently selected from H, monovalent C₁ to C₁₀-hydrocarbyl or a divalent C₁ to C₁₀-hydrocarbyl-A 1 group, wherein A1 is X2 or a terminating group selected from —COOH, —COORS, —CONH₂, —CON(R₅)2,, —OH, —SH, or —NH₂ wherein R₅ is monovalent C₁ to C₅-hydrocarbyl, preferably wherein R₂ₐ and R_{2b} are independently selected from H, —CH₃, —(CH₂).-CH₃, —(CH₂)_{n′}—COOH, —(CH₂)_{n′}—COOCH₃ or —(CH₂)_{n′}—COOCH₂CH₃ wherein n′ is from 1 to 3, more preferably —CH₂—CH₂—COOH or —CH₂—CH₂—COOCH₃, most preferably wherein R₂ₐ is —CH₂—CH₂—COOH or —CH₂—CH₂—COOCH₃.

8. The compound according to claim 1, wherein R₁ₐ and Rab are independently selected from H, —CH₃, —OH, —SH, or —NH₂, preferably H.

9. The compound according to claim 1, wherein R₄ is a C₁ to C₅-alkylene-A2 group, C₁ to C₅-ether-A2 group or C₁ to C₅-thioether-A2 group, wherein A2 is X3 or a terminating group selected from —COOH, —COOR₇, —CONH₂, —CON(R₅)₂, —OH, —SH, or —NH₂, wherein R₇ is monovalent C₁ to C₂-hydrocarbyl group, preferably —(CH₂)_{n″}—COOH or —(CH₂)_{n″}—COOCH₃ wherein n″ is from 1 to 3, more preferably —(CH₂)2—COOH or —(CH₂)2—COOCH₃, most preferably —(CH₂)₂—COOH.

10. The compound according to claim 1, wherein Mll+is ammonium or a metal cation, preferably ammonium or an iron, zinc or magnesium cation, more preferably an iron cation, most preferably an iron cation selected from iron(II) and iron(III).

11. The compound according to claim 1, wherein L is divalent C₁ to C₂₀-hydrocarbyl, preferably C₁ to C₂₀-alkylene, C₁ to C₂₀-ether, C₁ to C₂₀-aryl or C₁ to C₂₀-heteroaryl, more preferably C₁ to C₂₀-alkylene, C₁ to C₂₀-aryl or C₁ to C₂₀-heteroaryl comprising 1,4-phenylene and/or 1,2,3-triazole, for example 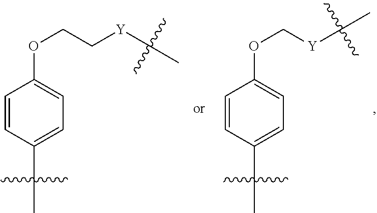
wherein Y is selected from O, NR₉ or 1,2,3-triazole, wherein R₉ is H or C₁ to C₆-hydrocarbyl.

12. The compound according to claim 1, wherein X is an aromatic or heteroaromatic compound optionally selected from a pyrene, anthracene, naphthalene, acridine, stilbene, indole, benzindole, oxazole, thiazole, thiazine, benzothiazole, cyanine, carbocyanine, salicylate, anthranilate, coumarin, fluorescein and/or rhodamine and derivatives thereof, preferably wherein X is selected from coumarin, fluorescein and derivatives thereof, indocyanine green and methylene blue and derivatives thereof.

13. The compound according to claim 1, wherein X and the tetrapyrrole or reduced tetrapyrrole backbone of the porphyrins, chlorins, bacteriochlorins or isobacteriochlorins represent a FRET pair.

14. The compound of claim 13 wherein fluorescence from X is quenched by FRET in the compound of any one of Formulas I to V following excitation of X, preferably wherein the compound is selected from porphyrins and X is coumarin or fluorescein or a derivative thereof and/or the compound is selected from chlorins, bacteriochlorins or isobacteriochlorins and X is indocyanine green or methylene blue or a derivative thereof.

15. The compound of claim 13 wherein the fluorescence from X is observed due to FRET in the compound of any one of Formulas I to Vh following excitation of the tetrapyrrole or reduced tetrapyrrole backbone of the porphyrins, chlorins, bacteriochlorins or isobacteriochlorins, preferably wherein the compound is selected from porphyrins and X is indocyanine green or methylene blue or a derivative thereof.

16. A compound according to claim 1 for use in a method of diagnosis, optionally of a disease characterised by heme oxygenase-1 (HO-1) over expression.

17. A compound according to claim 1 for use in a method of diagnosis in vivo of intraplaque haemorrhage, acute coronary syndrome and/or stroke caused by intraplaque haemorrhage and/or atherosclerosis.

18. A compound for use in a method of diagnosis according to claim 16, wherein the method comprises administering the compound to a subject, optionally wherein the compound is administered into the coronary artery in the diagnosis of acute coronary syndrome.

19. A compound for use in a method of diagnosis according to claim 16, wherein the method comprises providing light at a wavelength within the absorbance spectrum of X or the tetrapyrrole or reduced tetrapyrrole backbone of the porphyrins, chlorins, bacteriochlorins or isobacteriochlorins and detecting light emitted by X.

20. A compound according to claim 1 for use in a method of treatment of acute coronary syndrome and/or stroke caused by intraplaque haemorrhage and/or atherosclerosis.

21. A compound for use in a method of treatment according to claim 20, wherein the method comprises administering the compound to a subject, providing light at a wavelength within the absorbance spectrum of X or the tetrapyrrole or reduced tetrapyrrole backbone of the porphyrins, chlorins, bacteriochlorins or isobacteriochlorins, detecting light emitted by X and treating the location in the subject in which fluorescence modulation is observed.

22. A method for in vitro and/or ex vivo diagnosis of intraplaque haemorrhage, acute coronary syndrome and/or stroke caused by intraplaque haemorrhage and/or atherosclerosis using the compound according to claim 1.

23. The method of claim 22, wherein the method comprises contacting the compound with blood from a subject and/or providing light at a wavelength within the absorbance spectrum of X or the tetrapyrrole or reduced tetrapyrrole backbone of the porphyrins, chlorins, bacteriochlorins or isobacteriochlorins and detecting light emitted by X.

24. A method of imaging heme oxygenase-1 (HO-1) and/or intraplaque haemorrhage using a compound according to claim 1.

25. The method of claim 24, wherein the method comprises contacting the compound with a sample, and/or providing light at a wavelength within the absorbance spectrum of X or the tetrapyrrole or reduced tetrapyrrole backbone of the porphyrins, chlorins, bacteriochlorins or isobacteriochlorins and detecting light emitted by X.

26. Use of a compound according to claim 1 in the in vitro and/or ex vivo diagnosis of intraplaque haemorrhage, acute coronary syndrome and/or stroke caused by intraplaque haemorrhage and/or atherosclerosis.

27. Use of a compound according to claim 1 as contrast agent for imaging heme oxygenase-1 (HO-1) and/or intraplaque haemorrhage.

28. Use of a compound according to claim 1 as a research reagent, preferably for the detection of heme oxygenase-1 (HO-1).

29. A method of preparing a compound according to claim 1, comprising reacting a compound of Formula VIa to VIc or a pharmaceutically acceptable salt thereof: 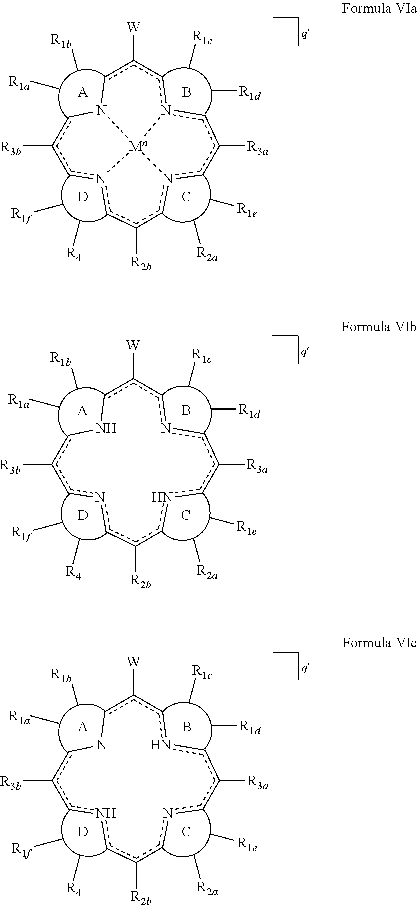
wherein the compound is a porphyrin, chlorin, bacteriochlorin or isobacteriochlorin, and wherein: [symbol]
represents a double or single bond; [symbol]
represents a dative or covalent bond between nitrogen and Mⁿ⁺;
R₁ₐ to R_{1f} are independently selected from H or monovalent hydrocarbyl; or i) R₁ₐ and R_{1b} are taken together to form a cyclocarbyl, ii) R_{1c} and R_{1d} are taken together to form a cyclocarbyl, iii) R₁ₑ is taken together with R₂ₐ to form a cyclocarbyl, and/or iv) R_{1f} is taken together with R₄ to form a cyclocarbyl;
R₂ₐ and R_{2b} are independently selected from H, monovalent hydrocarbyl or a divalent hydrocarbyl-A1 group, wherein A1 is X2 or a terminating group selected from —COOH, —CSOH, —COSH, —CSSH, —CONH₂, —OH, —SH, —NH₂, —COOR₇, —CSOR₅, —COSR₅, —CSSR₅, —CON(R₅)₂, —CONHR₅, —OR₅, —SR₅, —N(R₅)₂, —NHR₅, —CH(OR₅)₂, —CH(OR₅)(SR₅), —CH(SR₅)₂, 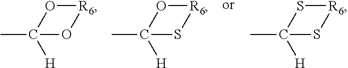
wherein R₅ is monovalent C₁ to C₅-hydrocarbyl or X2, and R₆ is C₁ to C₅ alkylene; or R₂ₐ is taken together with R₁ₑ to form a cyclocarbyl;
R₁ₐ and Rab are independently selected from H, —CH₃, —CH₂CH₃, —OH, —OCH₃, —OCOCH₃, —SH, —SCH₃, —NH₂, —NHCH₃, or —N(CH₃)₂;
R₄ is a divalent C₁ to C₁₀-hydrocarbyl-A2 group, or R₄ is taken together with R_{1f} to form a cyclocarbyl which is substituted by a divalent C₁ to C₁₀-hydrocarbyl-A2 group or by A2 alone, wherein A2 is X3 or a terminating group selected from —COOH, —CSOH, —COSH, —CSSH, —CONH₂, —OH, —SH, —NH₂, —COOR₇, —CSOR₇, —COSR₇, —CSSR₇, —CON(R₇)₂, —CONHR₇, —OR₇, —SR₇, —N(R₇)₂, —NHR₇ —CH(OR₇)₂, —CH(OR₇)(SR₇), —CH(SR₇)₂, 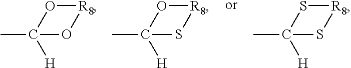
wherein R₇ is monovalent C₁ to C₅-hydrocarbyl or X3, and R₈ is C₁ to C₅-alkylene;
L is a linker;
X2 and X3 are fluorophores which may be the same or different;
Mⁿ⁺ is a cation having a positive charge of integer n, wherein n is from 1 to 3;
q′ is the overall charge of the compound;
A, B, C and D each independently represent a five-membered nitrogen-containing heterocycle selected from pyrrole, pyrroline or pyrrolidine rings; and
W comprises an azide or an alkyne functional group,
with a compound having the formula [Z-X]^{q′}, wherein X is a fluorophore which may be the same as or different from X2 and X3, q″ is the overall charge of the compound, and Z comprises an azide or an alkyne functional group,
wherein one of W and Z comprises an azide functional group and the other comprises an alkyne functional group.

30. The method of claim 29, wherein the reaction is performed in the presence of a copper-containing catalyst, preferably a catalyst comprising ascorbate and copper(I) and/or copper(II).

31. The method of claim 29, wherein W comprises divalent C₁ to C₂₀-hydrocarbyl terminated with an azide or an alkyne, preferably wherein the hydrocarbyl comprises a phenyl, and/or wherein Z is either an azide or an alkyne.
